(19) United States
(12) Patent Application Publication
(10) Pub. No.: US 2025/0207149 A1
(43) Pub. Date: Jun. 26, 2025

(54) A PRE-SCREENING METHOD AND A METHODS FOR SEPARATING ADENO-ASSOCIATED VIRUS CAPSIDS

(71) Applicant: CYTIVA BIOPROCESS R&D AB, Uppsala (SE)

(72) Inventors: Åsa Hagner McWhirter, Uppsala (SE); Jean-Luc Maloisel, Uppsala (SE); Brigitta Németh, Uppsala (SE)

(21) Appl. No.: 18/847,585

(22) PCT Filed: Apr. 26, 2023
(86) PCT No.: PCT/EP2023/060871

§ 371 Date: Sep. 16, 2024

(30) Foreign Application Priority Data
Apr. 27, 2022 (GB) 2206123.8

(51) Int. Cl. C12N 15/86; B01D 15/36; B01D 15/42; B01J 41/12

(52) U.S. Cl. CPC C12N 15/86; B01D 15/363; B01D 15/426; B01J 41/12; C12N 2750/14143; C12N 2750/14151

(57) ABSTRACT

The present disclosure is directed to a method for determining elution conditions suitable for separating adeno-associated virus (AAV) capsids fully packaged with genetic material from AAV capsids not fully packaged with genetic material, the method comprising: (a) adding a liquid sample comprising AAV capsids to a strong, or partially strong, anion exchange chromatography material comprising a surface extender, (b) eluting the AAV virus capsids from the chromatography material by applying an elution buffer comprising a step gradient of increasing conductivity, which increases by from about 0.5 to about 3 mS/cm per step, (c) based on an elution profile obtained in step (b), determining a first value of conductivity or conductivity-related parameter, which is suitable for eluting the adeno-associated virus capsids not fully packaged with genetic material, and (d) based on the elution profile obtained in step (b), determining a second value of conductivity or conductivity-related parameter, which is suitable for eluting the adeno-associated virus capsids fully packaged with genetic material. Further disclosed are methods for separating fully packaged AAV capsids from not fully packaged AAV capsids based on pre-determined first and second value of conductivity or conductivity-related parameter, as well as use of an anion exchange chromatography material for separating fully packaged AAV capsids from not fully packaged AAV capsids.

### FIELD OF THE DISCLOSURE

The present disclosure relates to the field of separation of adeno-associated capsids and is directed to a method for determining elution conditions suitable for separating adeno-associated virus capsids fully packaged with genetic material from adeno-associated virus capsids not fully packaged with genetic material. Further disclosed are methods for separating adeno-associated virus capsids fully packaged with genetic material from adeno-associated virus capsids not fully packaged with genetic material, and use of an anion exchange chromatography material for such separations. The present disclosure is applicable to separation of capsids of adeno-associated virus serotypes 1, 2, 3, 4, 5, 6, 7, 8, 9, and 10 (AAV1, AAV2, AAV3, AAV4, AAV5, AAV6, AAV7, AAV8, AAV9, and AAV10) and variants thereof.

### BACKGROUND OF THE DISCLOSURE

Adeno-associated viruses (AAV) are non-enveloped viruses that have linear single-stranded DNA (ssDNA) genome and that can be engineered to deliver DNA to target cells. Recombinant adeno-associated virus (rAAV) vectors have emerged as one of the most versatile and successful gene therapy delivery vehicles. There is an increasing demand to use viral vectors for gene therapy. To use AAV particles as vectors in therapy it is necessary to purify the virus particles from cell impurities like DNA after transfection. Ultracentrifugation is efficient but not scalable. Normally, several filtration steps and several chromatography steps are used to separate AAV particles from cell cultures (see e.g., Weihong Qu et al).

Therapeutic efficacy of AAV vectors is dependent on high percentage of virus particles fully packaged with genetic material of interest. Upstream expression systems deliver a mixture of fully packaged AAV particles (containing the genetic material of interest), empty AAV particles, and AAV particles which are partially packaged with genetic material of interest), together with impurities. There is thus a need to enrich fully packaged AAV particles in the purification process. However, there are several challenges in relation to achieving an efficient and scalable separation of fully packaged and empty adeno-associated virus capsids, such as:
Large diversity of capsids (serotypes and variants) and cell culture differences in terms of yield of full capsids, which means that extensive optimization is needed for purification of each serotype or variant of adeno-associated virus.
Small differences between fully packaged and empty capsids in relation to several parameters relevant for purification, e.g. isoelectric point;
In addition to fully packaged and empty capsids, also partially packaged capsid variants are produced in the infected host cells. There are indications that such partially packaged, and thereby therapeutically less effective, capsids may be partly co-eluted with fully packaged capsids.

Methods for separation of fully packaged capsids from not fully packaged capsids have been previously described (see e.g., Hejmowski et al). However, further optimisation of purification strategies is always desired to increase the speed and decrease the cost of downstream processing of different adeno-associated virus serotypes.

### SUMMARY OF THE DISCLOSURE

The object of the present disclosure is to provide an improved method for separation of fully packaged adeno-associated virus capsids from not fully packaged adeno-associated virus capsids. This is achieved by first performing a method for determining elution conditions suitable for separating fully packaged capsids from not fully packaged capsids. Herein, this method is called a pre-screening method. It is followed by performing a method for separating fully packaged from not fully packaged capsids by eluting not fully packaged capsids at a first conductivity value as determined in the pre-screening method, and by eluting fully packaged capsids at a second conductivity value as determined in the pre-screening method. Thereby, an improved resolution between fully packaged and not fully packaged capsids is obtained, which results in achieving a composition having a higher ratio of fully packaged capsids to not fully packaged capsids. The focus of the disclosure is a pre-screening method for establishing optimal elution conditions for the polishing step of a separation method, also called secondary or final purification.

More particularly, a first aspect of the present disclosure is directed to the so-called pre-screening method, which is a method for determining elution conditions suitable for separating adeno-associated virus (AAV) capsids fully packaged with genetic material from AAV capsids not fully packaged with genetic material, the method comprising:
(a) adding a liquid sample comprising AAV capsids to a strong, or partially strong, anion exchange chromatography material comprising a surface extender,
(b) eluting the AAV virus capsids from the chromatography material by applying an elution buffer comprising a step gradient of increasing conductivity, which increases by from about 0.5 to about 3 mS/cm per step,
(c) based on an elution profile obtained in step (b), determining a first value of conductivity or conductivity-related parameter, which is suitable for eluting the adeno-associated virus capsids not fully packaged with genetic material, and
(d) based on the elution profile obtained in step (b), determining a second value of conductivity or conductivity-related parameter, which is suitable for eluting the adeno-associated virus capsids fully packaged with genetic material.

Even more particularly, the pre-screening method is a method for determining elution conditions suitable for separating adeno-associated virus capsids fully packaged with genetic material from adeno-associated virus capsids not fully packaged with genetic material, the method comprising the following steps:
a) adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, to a strong, or partially strong, anion exchange chromatography material comprising a support and a ligand for binding to the adeno-associated virus capsids, wherein the chromatography material comprises a surface extender connecting the ligand to the support, wherein the surface extender is a polymer, wherein the polymer is selected from:
   (i) a polymer having a naturally occurring skeleton, such as a polysaccharide, such as starch, cellulose, dextran, or agarose, and
   (ii) a polymer having a synthetic skeleton, such as a polyvinyl alcohol, a polyacrylamide, a polymethacrylamide, or a polyvinyl ether;
b) eluting the adeno-associated virus capsids not fully packaged with genetic material and the adeno-associated virus capsids fully packaged with genetic material from the chromatography material by applying an elution buffer comprising a step gradient of increasing conductivity, which starts at from about 0 to about 5 mS/cm, and which increases by from about 0.5 to about 3 mS/cm per step, such as from about 1 to about 2 mS/cm per step, such as from about 1.2 to about 1.5 mS/cm per step, at least up to and including a conductivity at which the adeno-associated virus capsids not fully packaged with genetic material and the adeno-associated virus capsids fully packaged with genetic material have been eluted from the chromatography material;
c) based on an elution profile obtained in step (b), determining a first value of conductivity or conductivity-related parameter, which is suitable for eluting the adeno-associated virus capsids not fully packaged with genetic material, and
d) based on the elution profile obtained in step (b), determining a second value of conductivity or conductivity-related parameter, which is suitable for eluting the adeno-associated virus capsids fully packaged with genetic material.

The present disclosure further provides a method for separating adeno-associated virus capsids fully packaged with genetic material from adeno-associated virus capsids not fully packaged with genetic material, the method comprising steps (a)-(d) of the pre-screening method described above, and further comprising the steps:
e. adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, the liquid sample originating from a cell culture harvest from which the liquid sample of step (a) originated, to the chromatography material as defined in step (a);
f. eluting the adeno-associated virus capsids not fully packaged with genetic material by applying an elution buffer having the first value of conductivity or conductivity-related parameter as determined in step (c); and
g. eluting the adeno-associated virus capsids fully packaged with genetic material by applying an elution buffer having the second value of conductivity or conductivity-related parameter as determined in step (d);
wherein
   (i) the duration of step (f) is at least 3 times, such as 4 times, the duration of step (g), and/or
   (ii) the method comprises a step (f′) between step (f) and step (g), wherein step (f′) comprises applying an additional step elution and/or a gradient of increasing conductivity between the first value and the second value of conductivity or conductivity-related parameter, and the duration of steps (f) and (f) is at least 3 times, such as 4 times, the duration of step (g).

Additionally, the present disclosure is directed to a method for separating adeno-associated virus capsids fully packaged with genetic material from adeno-associated virus capsids not fully packaged with genetic material, the method comprising:
(I) adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, to a strong, or partially strong, anion exchange chromatography material comprising a support and a ligand for binding to the adeno-associated virus capsids, wherein the chromatography material comprises a surface extender connecting the ligand to the support, wherein the surface extender is a polymer, wherein the polymer is selected from:
   (i) a polymer having a naturally occurring skeleton, such as a polysaccharide, such as starch, cellulose, dextran, or agarose, and
   (ii) a polymer having a synthetic skeleton, such as a polyvinyl alcohol, a polyacrylamide, a polymethacrylamide, or a polyvinyl ether;
(II) eluting the adeno-associated virus capsids not fully packaged with genetic material by applying an elution buffer having a pre-determined first value of conductivity or conductivity-related parameter;
(III) eluting the adeno-associated virus capsids fully packaged with genetic material by applying an elution buffer having a pre-determined second value of conductivity or conductivity-related parameter;
the pre-determined first and second value of conductivity or conductivity-related parameter having been determined during separation of a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, the liquid sample originating from a cell culture harvest from which the liquid sample of step (I) originates, optionally having been determined by performing the method of steps (a)-(d) of the above-described pre-screening method;
wherein
   (i) the duration of step (II) is at least 3 times, such as 4 times, the duration of step (III), and/or
   (ii) the method comprises a step (II′) between step (II) and step (III), wherein step (II′) comprises applying a step elution and/or a gradient of increasing conductivity between the first value and the second value of conductivity or conductivity-related parameter, and the duration of steps (II) and (II′) is at least 3 times, such as 4 times, the duration of step (III).

The present disclosure also provides use of an anion exchange chromatography material comprising a support, a ligand, and a surface extender connecting the ligand to the support, and being defined by Formula IV:

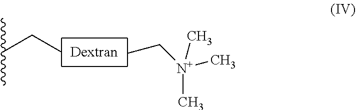

for separating adeno-associated virus capsids fully packaged with genetic material from adeno-associated virus capsids not fully packaged with genetic material, comprising determining elution conditions suitable for separating adeno-associated virus capsids
fully packaged with genetic material from adeno-associated virus capsids not fully packaged with genetic material,
wherein said elution conditions are determined by performing the steps:
a. adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, to the chromatography material;
b. eluting the adeno-associated virus capsids not fully packaged with genetic material and the adeno-associated virus capsids fully packaged with genetic material from the chromatography material by applying an elution buffer comprising a step gradient of increasing conductivity, which starts at from about 0 to about 5 mS/cm, and which increases by from about 0.5 to about 3 mS/cm per step, such as from about 1 to about 2 mS/cm per step, such as from about 1.2 to about 1.5 mS/cm per step, at least up to and including a conductivity at which the adeno-associated virus capsids not fully packaged with genetic material and the adeno-associated virus capsids fully packaged with genetic material have been eluted from the chromatography material;
c. based on an elution profile obtained in step (b), determining a first value of conductivity or conductivity-related parameter, which is suitable for eluting the adeno-associated virus capsids not fully packaged with genetic material, and
d. based on the elution profile obtained in step (b), determining a second value of conductivity or conductivity-related parameter, which is suitable for eluting the adeno-associated virus capsids fully packaged with genetic material.

Preferred aspects of the present disclosure are described below in the detailed description and in the dependent claims.

### BRIEF DESCRIPTION OF THE DRAWINGS

FIG. 1 is a flow chart of a pre-screening method, i.e., a method for determining elution conditions suitable for separating adeno-associated virus capsids according to the present disclosure.

FIG. 2 is a flow chart of a method for separating adeno-associated virus capsids comprising steps (a)-(d) according to the pre-screening method of FIG. 1 and further comprising steps (e)-(g) according to the present disclosure.

FIG. 3 is a flow chart of a method for separating adeno-associated virus capsids comprising applying pre-determined values of conductivity according to the present disclosure.

FIG. 4 is a graph showing the elution curve for fully packaged and empty capsids according to a separation method as described in Example 1 below.

FIG. 5 is a graph showing the elution curve for fully packaged and empty capsids according to a separation method as described in Example 1 below.

FIG. 6 is a graph showing the elution curve for fully packaged and empty capsids according to a separation method as described in Example 1 below.

FIG. 7 is a graph showing the elution curve for fully packaged and empty capsids according to a separation method as described in Example 2 below.

### DETAILED DESCRIPTION OF THE DISCLOSURE

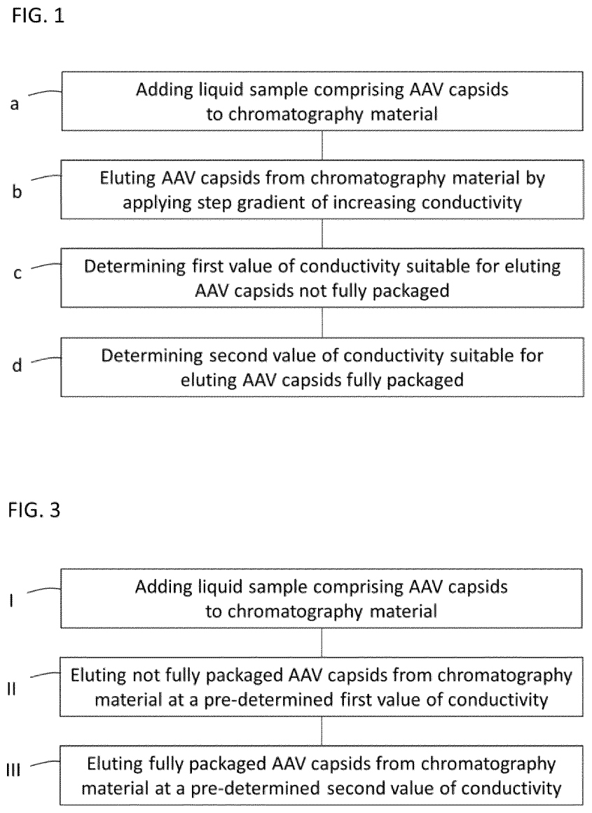

The present disclosure solves or at least mitigates the problems associated with existing methods for separating fully packaged adeno-associated virus capsids from capsids not fully packaged adeno-associated virus capsids by providing, as illustrated in FIG. 1, a pre-screening method, i.e., a method for determining elution conditions suitable for separating adeno-associated virus capsids fully packaged with genetic material from adeno-associated virus capsids not fully packaged with genetic material, the method comprising the following steps:
a) adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, to a strong, or partially strong, anion exchange chromatography material comprising a support and a ligand for binding to the adeno-associated virus capsids, wherein the chromatography material comprises a surface extender connecting the ligand to the support, wherein the surface extender is a polymer, wherein the polymer is selected from:
   (i) a polymer having a naturally occurring skeleton, such as a polysaccharide, such as starch, cellulose, dextran, or agarose, and
   (ii) a polymer having a synthetic skeleton, such as a polyvinyl alcohol, a polyacrylamide, a polymethacrylamide, or a polyvinyl ether;
b) eluting the adeno-associated virus capsids not fully packaged with genetic material and the adeno-associated virus capsids fully packaged with genetic material from the chromatography material by applying an elution buffer comprising a step gradient of increasing conductivity, which starts at from about 0 to about 5 mS/cm, and which increases by from about 0.5 to about 3 mS/cm per step, such as from about 1 to about 2 mS/cm per step, such as from about 1.2 to about 1.5 mS/cm per step, at least up to and including a conductivity at which the adeno-associated virus capsids not fully packaged with genetic material and the adeno-associated virus capsids fully packaged with genetic material have been eluted from the chromatography material;
c) based on an elution profile obtained in step (b), determining a first value of conductivity or conductivity-related parameter, which is suitable for eluting the adeno-associated virus capsids not fully packaged with genetic material, and
d) based on the elution profile obtained in step (b), determining a second value of conductivity or conductivity-related parameter, which is suitable for eluting the adeno-associated virus capsids fully packaged with genetic material.

Herein, it is shown that the currently disclosed pre-screening method is a universal way of dealing with the weaknesses of previously applied methods, in which differences in ionic capacity, anion exchange ligand density, amount of surface extender, and support material between different lots of chromatography materials, as well as feed variability and variations in buffer preparations, makes it nearly impossible to predict the amount of conductivity that is needed to achieve baseline separation of full and empty capsids.

A “virus particle” is herein used to denote a complete infectious virus particle. It includes a core, comprising the genome of the virus (i.e., the viral genome), either in the form of ribonucleic acid (RNA) or deoxyribonucleic acid (DNA), and the core is surrounded by a morphologically defined shell. The shell is called a capsid. The capsid and the enclosed viral genome together constitute the so-called nucleocapsid. The nucleocapsid of some viruses is surrounded by a lipoprotein bilayer envelope. In the field of bioprocessing, for the purpose of producing viral vectors for various applications such as therapy, the genome of a virus particle is modified to include a genetic insert, comprising genetic material of interest. Modified virus particles are allowed to infect host cells in a cell culture and the virus particles are propagated in said host cells, after which the virus particles are purified from the cell culture by any means of separation and purification. Herein, a virus particle to be separated from a cell culture by the presently disclosed method may alternatively be referred to as a “target molecule” or “target”. It is to be understood that “a virus particle” is intended to mean a type of virus particle and that the singular form of the term may encompass a large number of individual virus particles. Herein, the term “virus particle” may be used interchangeably with the terms “vector” and “capsid”, respectively, as further defined below.

The term “vector” is herein used to denote a virus particle, normally a recombinant virus particle, which is intended for use to achieve gene transfer to modify specific cell type or tissue. A virus particle can for example be engineered to provide a vector expressing therapeutic genes. Several virus types are currently being investigated for use to deliver genetic material (e.g., genes) to cells to provide either transient or permanent transgene expression. These include adenoviruses, retroviruses (γ-retroviruses and lentiviruses), poxviruses, adeno-associated viruses (AAV), baculoviruses, and herpes simplex viruses. Herein, the term “vector” may be used interchangeably with the terms “virus particle” and “capsid”, respectively.

The term “capsid” means the shell of a virus particle. The capsid surrounds the core of the virus particle, and normally should comprise a viral genome. A modified (recombinant) capsid, as produced in an upstream process of manufacturing, is supposed to comprise a complete viral genome, which genome includes genetic material of interest for one or more applications, for example of interest for various therapeutic applications. However, owing to low packaging efficiency, assembled capsids do not always contain any genetic material or only encapsidate truncated genetic fragments, resulting in so-called empty capsids and partially filled capsids, respectively. These capsids possess no therapeutic function, yet they compete for binding receptors during the cell-mediated processes. This may diminish the overall therapeutic efficacy and trigger undesirable immune responses. As a result, tracking these capsids throughout the production process is crucial to ensure consistent product quality and a proper dosing response (Xiaotong Fu et al). In up to 20-30% of a population of virus particles artificially produced in a cell culture, the capsid is only partially filled with genetic material. Further, in up to as much as 98% of artificially produced virus particles, the capsid does not comprise any part of the viral genome at all, i.e., it is empty. However, generally between 80% to 90% of artificially produced virus particles have empty capsids, and best cases currently achieve as little as 50% empty capsids.

Herein, the term “capsid” may be used interchangeably with the terms “vector” and “virus particle”, respectively. In the context of the present disclosure, a capsid may or may not comprise genetic material.

The term “genetic material of interest” is intended to mean genetic material which in the field of bioprocessing is considered relevant and valuable to get produced by viral replication and to purify such that it can be used in various applications, such as, but not limited to, therapeutic applications. As a non-limiting example, genetic material of interest may comprise a therapeutically relevant genetic material, such as a therapeutically relevant nucleotide sequence.

The term “capsid fully packaged with genetic material” is herein used to denote a capsid which has been correctly produced (by the host cell), or in other words,
a capsid which comprises a complete viral genome, or in other words,
a capsid comprising 100% of its viral genome, or in other words,
a capsid comprising a functional viral genome.

The viral genome includes a genetic insert, comprising genetic material of interest, as defined elsewhere herein.

A capsid which comprises a complete viral genome may herein alternatively be called a “full capsid” or a “fully packaged capsid”. The terms “full capsid”, “fully packaged capsid”, and “capsid fully packaged with genetic material” may be used interchangeably throughout this text.

The term “capsid not fully packaged with genetic material” is herein used to denote a capsid which has not been correctly produced (by the host cell), or in other words,
a capsid which does not comprise a complete viral genome, or in other words,
a capsid which comprises less than 100% of its viral genome.

A capsid which is not fully packaged with genetic material is either partially filled with genetic material or is not filled with any genetic material at all.

The term “capsid not fully packaged with genetic material” encompasses the terms “partially filled capsid” and “empty capsid”, as defined below.

A “partially filled capsid” is herein defined as a capsid which comprises parts of its viral genome, such as defective parts of its viral genome, or in other words,
a capsid which comprises a partial viral genome, or in other words,
a capsid which comprises a non-complete viral genome, or in other words,
a capsid which comprises a defective viral genome, or in other words,
a capsid which comprises more than 0% and less than 100% of the complete viral genome, such as from about 1% to about 99%, such as from about 5% to about 95%, such as from about 10% to about 90%, or such as about 1%, about 5%, about 10%, about 20%, about 30%, about 40%, about 50%, about 60%, about 70%, about 80%, about 90%, about 95%, or about 99%, of the complete viral genome. Since a partially filled capsid is an incorrectly produced capsid, it is desirable to separate and remove as many as possible of the partially filled capsids from a population of capsids, before putting the population of capsids to use in its intended application, e.g., a therapeutic application. Herein, a partially filled capsid may alternatively be called an “intermediate capsid”.

An “empty capsid” is herein defined as a capsid which does not comprise any part of its viral genome, i.e., which comprises 0% of its viral genome, or in other words, a capsid which is not filled with any genetic material at all. Thus, an empty capsid does not comprise any genetic material of interest. Consequently, it is desirable (and sometimes required, e.g., due to clinical regulations) to separate and remove as many as possible of the empty capsids from a population of capsids, before putting the population of capsids to use in its intended application, e.g., a therapeutic application.

Before putting a population of virus particles to use in its intended application, e.g., a therapeutic application, it is desirable (sometimes even required, e.g., due to clinical regulations) to enrich the full capsids, i.e., to increase the percentage of full capsids at the expense of the percentage of partially filled capsids and empty capsids.

The percentage of full capsids and empty capsids in a population of capsids can be estimated or analyzed with several methods known in the art. Some of these methods are briefly described below:

1: A260:280 in chromatogram will give an estimation of percentage full capsids present in peaks (ratio 1-1.5 indicate enriched in full capsids, ratio 0.5-0.7 is containing mainly empty capsids).

2. qPCR:ELISA ratio. qPCR quantifies viral genomes and ELISA quantifies total viral particles. A ratio of 2 assays with variation is less accurate and will be uncertain. Requires orthogonal analysis for confirmation (see below, 3,4 or 5).

3. Analytical anion exchange separating full and empty capsids (A260:280 ratio and peak area to calculate the percentage). Accuracy dependent of peak definition.

4. Analytical ultracentrifugation (AUC). Detects and quantifies particles of different density (corresponding to full, partially filled, and empty capsids). This is currently known as the “golden standard” in the art. However, ultracentrifugation is not scalable and thus is not suitable for analysis of large-scale batches of capsids.

5. Transmission electron microscopy (TEM). Image analysis counting particles (full, partially filled, and empty capsids). May introduce artifacts from sample preparation.

Some methods for estimating or analyzing the percentage of full capsids and empty capsids in a population of capsids are described in more detail in Xiaotong Fu et al, which is hereby incorporated by reference herein.

It is to be understood that the term “liquid sample” as used herein encompasses any type of sample obtainable from a cell culture, or from a fluid originating from a cell culture which fluid is at least partly purified, by any means of separation and purification.

The term “separation matrix” is used herein to denote a material comprising a support to which one or more ligands comprising functional groups have been coupled. The functional groups of the ligand(s) bind compounds herein also called analytes, which are to be separated from a liquid sample and/or which are to be separated from other compounds present in the liquid sample. A separation matrix may further comprise a compound which couples the ligand(s) to the support. The terms “linker”, “extender”, and “surface extender” may be used to describe such a compound, as further described below. The term “resin” is sometimes used for a separation matrix in this field. The terms “chromatography material” and “chromatography matrix” are used herein to denote a type of separation matrix.

The term “surface” herein means all external surfaces and includes in the case of a porous support outer surfaces as well as pore surfaces.

Herein, the term “strong anion exchange chromatography material” is intended to mean a chromatography material which comprises a ligand comprising a quaternized amine group. A quaternary amine group is a strong anion exchange group, which is always positively charged irrespective of to which pH it is subjected. For DEAE-based types of chromatography materials, the degree of quaternization of the amine group may vary among the amine groups included in a chromatography material. A degree of quaternization of the amine group of from about 12% to about 100% globally in a chromatography material is generally considered to result in a chromatography material which behaves like a strong, or at least partially strong, anion exchange chromatography material since these at least 12% of all amine groups are always charged. In contrast to quaternized amine groups, almost all other ionic exchange groups are weak, i.e., their charge varies from fully charged to not charged within a reasonable range of pH used (such as pH 2-11) and having a neutral charge (same amount of + and − charges) at pl.

Capto Q (Cytiva, Sweden) is a non-limiting example of a strong anion exchange chromatography material having about 100% quaternized amine groups. Capto DEAE (Cytiva, Sweden) is a non-limiting example of a strong, or partially strong, anion exchange chromatography material having a degree of quaternization of the amine groups of about 15%.

The separation matrix may be contained in any type of separation device, as further defined elsewhere herein. As a non-limiting example, a chromatography material may be packed in a chromatography column, before adding a liquid sample to the chromatography material being contained in the chromatography column. As another example, for the present pre-screening method, the chromatography material may be provided in a multi-well format, such as in the form of a multi-well plate having wells containing the chromatography material (e.g. PreDictor Capto Q plates, Cytiva, Sweden).

In this context, “ligand” is a molecule that has a known or unknown affinity for a given analyte and includes any functional group, or capturing agent, immobilized on its surface, whereas “analyte” includes any specific binding partner to the ligand. The term “ligand” may herein be used interchangeably with the terms “specific binding molecule”, “specific binding partner”, “capturing molecule” and “capturing agent”. Herein, the molecules in a liquid sample which interact with a ligand are referred to as “analyte”. The analytes of interest according to the present disclosure are adeno-associated virus capsids, more particularly adeno-associated virus capsids either fully packaged or not fully packaged with genetic material. Consequently, herein the terms “analyte”, “adeno-associated virus capsid” and “capsid” may be used interchangeably.

In the herein disclosed method for separating fully packaged capsids from not fully packaged capsids, the chromatography material used comprises a linker connecting the ligand to the support, i.e., the coupling of the ligand to the support is provided by introducing a linker between the support and ligand. The coupling may be carried out following any conventional covalent coupling methodology such as by use of epichlorohydrin; epibromohydrin; allyl-glycidylether; bis-epoxides such as butanedioldiglycidylether; halogen-substituted aliphatic substances such as di-chloro-propanol; and divinyl sulfone. Other non-limiting examples of suitable linkers are: polyethylene glycol (PEG) having 2-6 carbon atoms, carbohydrates having 3-6 carbon atoms, and polyalcohols having 3-6 carbon atoms. These methods are all well known in the art and easily carried out by the skilled person.

The ligand is coupled to the support via a longer linker molecule, also known as a “surface extender”, or simply “extender”. Extenders are well known in this field, and commonly used to sterically increase the distance between ligand and support. Extenders are sometimes denoted tentacles or flexible arms. For a more detailed description of possible chemical structures, see for example U.S. Pat. No. 6,428,707, which is hereby included herein by reference. In brief, the extender may be in the form of a polymer such as a homo- or a copolymer. Hydrophilic polymeric extenders may be of synthetic origin, i.e., with a synthetic skeleton, or of biological origin, i.e., a biopolymer with a naturally occurring skeleton. Typical synthetic polymers are polyvinyl alcohols, polyacryl- and polymethacrylamides, polyvinyl ethers etc. Typical biopolymers are polysaccharides, such as starch, cellulose, dextran, agarose. Extenders may be linear and non-linear (branched) polymers, such as a brush polymer, which is a long linear structure with functional appendices along its length. The results described in the Examples herein surprisingly show that a chromatography material comprising a surface extender provides an improved separation of full AAV capsids from empty AAV capsids compared to the same chromatography material not including a surface extender.

The term “eluent” is used in its conventional meaning in this field, i.e., a buffer of suitable pH and/or ionic strength to release one or more compounds from a separation matrix.

The term “eluate” is used in its conventional meaning in this field, i.e., the part(s) of a liquid sample which are eluted from a chromatography column after having loaded the liquid sample onto the chromatography column.

As mentioned above, in the method for determining elution conditions suitable for separating fully packaged capsids from not fully packaged capsids, the liquid sample which is added to a chromatography material in step (a) comprises adeno-associated virus capsids of a purity of at least 90%, such as 90%, 91%, 92%, 93%, 94%, 95%, 96%, 97%, 98%, or 99% and of a concentration of at least 10¹², such as 10¹³, 10¹⁴, or 10¹⁵, adeno-associated virus capsids/ml, of which at least 5%, such as 6%, 7%, 8%, 9%, 10%, 15%, 20%, 25%, 30%, 40%, 50%, 60%, 70%, or 80%, of the adeno-associated virus capsids are adeno-associated virus capsids fully packaged with genetic material. With regard to the purity of adeno-associated capsids in the liquid sample, a purity of at least 90%, such as up to 99%, is intended to mean that at least 90%, such as up to 99%, of the biological material in the liquid sample is represented by adeno-associated capsids (including full, empty, and partially filled capsids) while the remaining up to 10%, such as 1%, is represented by host cell protein and DNA.

As specified in step (b) of the above-described pre-screening method, a step gradient elution is designed in the form of a stepwise slowly increasing conductivity, which starts at from about 0 to about 5 mS/cm, such as at about 0, 0.5, 1.0, 1.5, 2.0, 2.5, 3.0, 3.5, 4.0, 4.5, or 5.0 mS/cm. The step gradient increases by approx. 0.5-3 mS/cm increase per step, such as from about 1 to about 2 mS/cm per step, such as from about 1.2 to about 1.5 mS/cm per step, or by about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mS/cm per step, at least up to and including a conductivity at which the capsids not fully packaged with genetic material and the capsids fully packaged with genetic material have been eluted from the chromatography material, in order to identify which value of conductivity is needed to wash out empty capsids and full capsids, respectively. It is to be understood that the conductivity step gradient may optionally increase above the conductivity at which both empty and full capsids have been eluted.

As specified in steps (c) and (d) of the above-described pre-screening method, a first and second value of conductivity or conductivity-related parameter are determined based on an elution profile obtained in step (b). The elution profile may be in the form of a chromatogram, or a table or a graph comprising elution-related values.

The term “conductivity-related parameter” as used herein is intended to mean a parameter which influences the conductivity of a solution. A conductivity-related parameter may for example be directly correlated or inversely correlated with the conductivity. Non-limiting examples of conductivity-related parameters which may be relevant in this context are salt concentration and pH, as well as presence/concentration of compounds improving the separation between capsids fully packaged with genetic material and capsids not fully packaged with genetic material. Compounds which improve separation may for example be selected from a carbohydrate, a divalent metal ion, and a detergent, as described in detail further below.

The first value of conductivity or conductivity-related parameter determined shall be suitable for eluting the adeno-associated virus capsids not fully packaged with genetic material, and the second value of conductivity or conductivity-related parameter determined shall be suitable for eluting the adeno-associated virus capsids fully packaged with genetic material. In a pre-screening method where empty capsids are eluted before full capsids, the first value of conductivity is normally determined to be the value of conductivity applied when eluting the first peak containing empty and/or full capsids. If the empty capsids do not bind to the column but end up in the flowthrough, the first value of conductivity will be the same as the baseline conductivity value, i.e., the conductivity before the step gradient of conductivity is applied. Accordingly, in some instances the first value of conductivity may be determined to be as low as 0 mS/cm.

In a pre-screening method where empty capsids are eluted before full capsids, the second value of conductivity is normally determined to be a value of conductivity equal to or higher than the conductivity value applied when eluting the last peak containing empty and/or full capsids. For example, if the last peak is eluted at a conductivity value of 5 mS/cm, the second value of conductivity is determined to be ≥5 mS/cm.

The elution buffer applied in step (b) of the pre-screening method may comprise a salt. The step gradient of increasing conductivity in step (b) of the pre-screening method may be a step gradient of increasing salt concentration. In this context, the conductivity-related parameter referred to in steps (c) and (d) of the pre-screening method may be the salt concentration. The salt may be a kosmotropic salt. Salts in water solvent are defined as kosmotropic (order-making) if they contribute to the stability and structure of water-water interactions. In contrast, chaotropic (disorder-making) salts have the opposite effect, disrupting water structure, increasing the solubility of nonpolar solvent particles, and destabilizing solute aggregates. Kosmotropes cause water molecules to favorably interact, which in effect stabilizes intramolecular interactions in macromolecules such as proteins (Moelbert S et al). A scale can be established for example by referring to the Hofmeister series, or lyotropic series, which is a classification of ions in order of their ability to salt out or salt in proteins (Hyde A et al).

More particularly, the salt may comprise (i) an anion selected from a group consisting of CO₃²⁻, SO₄²⁻, S₂O₃²⁻, H₂PO₄⁻, HPO₄²⁻, acetate⁻, citrate⁻, and Cl⁻, and (ii) a cation selected from a group consisting of NH₄⁺, K⁺, Na⁺, and Li⁺. In a currently preferred embodiment, the salt is sodium acetate (NaOAc). Non-limiting examples of suitable concentrations of NaOAc include from about 5 mM to about 500 mM, such as about 5, 10, 20, 30, 40, 50, 60, 70, 80, 90, 100, 150, 200, 250, 300, 350, 400, 450, or 500 mM. However, it is to be understood that other salts consisting of a combination an anion as listed under (i) and a cation as listed under (ii) may alternatively be used to elute the capsids. Non-limiting examples of such other salts are NaCl, LiCl, KCl, or other equivalent metal salt suitable to use for salt elution, as is well known in the art. Non-limiting examples of suitable concentrations of NaCl include from about 5 mM to about 2M, such as about 5, 10, 20, 30, 40, 50, 100, 150, 200, 250, 300, 350, 400, 450, 500, 600, 700, 800, 900, 1000, 1500, or 2000 mM.

Step (b) of the pre-screening method may comprise adding a volume of the elution buffer corresponding to from about 1 to about 10 volumes, such as from about 2 to about 8 volumes, such as about 5 volumes, or about 1, 2, 3, 4, 5, 6, 7, 8, 9, or 10 volumes, of the chromatography material, per step of the step gradient.

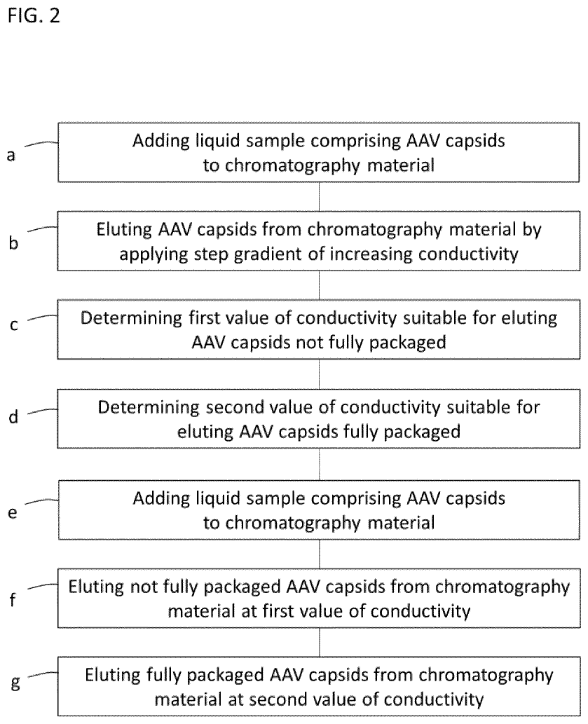

The above-described pre-screening method is followed by a two-step elution method, which is designed based on the information that is provided by the elution profile (e.g., chromatogram) obtained in the pre-screening method. More particularly, the present disclosure further provides, as illustrated in FIG. 2, a method for separating fully packaged adeno-associated virus capsids from not fully packaged adeno-associated virus capsids, comprising performing steps (a)-(d) of the pre-screening method as described in detail above, the method further comprising, steps (e)-(g) as follows:
e. adding a liquid sample comprising adeno-associated virus capsids of a purity of at least 90% and of a concentration of at least 10¹² adeno-associated virus capsids/ml, of which at least 5%, such as 6%, 7%, 8%, 9%, 10%, 15%, 20%, 25%, 30%, 40%, 50%, 60%, 70%, or 80%, of the adeno-associated virus capsids are adeno-associated virus capsids fully packaged with genetic material, the liquid sample originating from a cell culture harvest from which the liquid sample of step (a) originated, to the chromatography material as defined in step (a);
f. eluting the adeno-associated virus capsids not fully packaged with genetic material by applying an elution buffer having the first value of conductivity or conductivity-related parameter as determined in step (c); and
g. eluting the adeno-associated virus capsids fully packaged with genetic material by applying an elution buffer having the second value of conductivity or conductivity-related parameter as determined in step (d);
wherein
   (i) the duration of step (f) is at least 3 times, such as 4 times, the duration of step (g), and/or
   (ii) the method comprises a step (f′) between step (f) and step (g), wherein step (f′) comprises applying an additional step elution and/or a gradient of increasing conductivity between the first value and the second value of conductivity or conductivity-related parameter, and the duration of steps (f) and (f′) is at least 3 times, such as 4 times, the duration of step (g).

The liquid sample added in step (e) should originate from the same cell culture harvest as the liquid sample of step (a) of the pre-screening method, in order that the elution conditions determined in the pre-screening method are surely applicable also to the liquid sample added in step (e).

The aim of steps (f) and (g) of the above-disclosed method is to obtain fully packaged capsids of a purity which is as high as possible. A person skilled in the art readily understands that this may be achieved by applying various different separation conditions. Non-limiting examples of separation conditions to obtain fully packaged capsids of a purity as high as possible include separation conditions which allow binding of not fully packaged capsids to the chromatography material, while:
(i) allowing fully packaged capsids to substantially flow through the chromatography material (i.e., fully packaged capsids substantially not binding to the chromatography material), or
(ii) allowing fully packaged capsids to bind to the chromatography material followed by eluting them from the chromatography material. It is to be understood that in the bind-elute process described in item (ii), the fully packaged capsids may be eluted from the chromatography material before or after not fully packaged capsids, depending on which separation conditions are applied.

As mentioned above, there are small differences between fully packaged capsids and not fully packaged capsids in relation to several parameters relevant for purification, e.g., their isoelectric point. This often leads to (at least partial) co-elution of fully packaged and not fully packaged capsids. Accordingly, realistically, the adeno-associated virus capsids eluted in step (b) of the above-disclosed method will not be completely separated into full, empty, and partially filled capsids. However, there will be eluate fractions which comprise a substantially higher percentage of full capsids than in the liquid sample added to the chromatography material in step (e). More particularly, the adeno-associated virus capsids eluted in step (g), i.e., adeno-associated virus capsids fully packaged with genetic material, may be eluted into eluate fractions, which eluate fractions combined comprise at least 50%, such as 55%, 60%, 65%, 70%, 75%, 80%, 85%, or 90%, of the adeno-associated virus capsids of the liquid sample added in step (e), of which at least 60%, such as 65%, 70%, 75%, 80%, 85%, or 90%, of the adeno-associated virus capsids are fully packaged with genetic material. Non-limiting examples of recovery and purification of full capsids achieved by the presently disclosed method are a recovery of at least 50% of the capsids of the liquid sample added in step (e), of which at least 60% are full capsids, such as a recovery of at least 70% of the capsids of the liquid sample added in step (a), of which at least 80% are full capsids. In Example 1 described further below, the results show a recovery of at least 80% of viral genomes from harvest, of which at least 70% are full capsids.

It has been found advantageous to perform step (f) for a duration of time which is at least 3 times, such as 4 times, 5 times or more, compared to the duration of step (g). Without wishing to be bound by theory, it is believed that the relatively longer duration of step (f) is beneficial or even crucial for eluting substantially all, or nearly all, of the empty capsids present in the liquid sample.

An alternative to performing step (f) for a duration at least 3 times the duration of step (g), the method may comprise applying an additional step (f′) between step (f) and step (g), wherein the duration of steps (f) and (f′) is at least 3 times, such as 4 times, 5 times or more, compared to the duration of step (g). Step (f′) may for example comprise:
(i) an elution step performed at a value of conductivity or conductivity-related parameter between the first and second value of conductivity or conductivity-related parameter,
(ii) a linear gradient of increasing conductivity between the first and second value of conductivity or conductivity-related parameter, or
(iii) a combination of (i) and (ii).

The difference in duration between step (f) and step (g) may be accomplished by adding a volume of the elution buffer in step (f), which is at least 3 times, such as 4 times or more, higher than the volume of elution buffer added in step (g). As a non-limiting example, in step (f) a volume of elution buffer corresponding to from about 3 to about 30 volumes, such as from about 6 to about 24 volumes, such as about 15 volumes, or about 3, 4, 5, 6, 7, 8, 9, 10, 11, 12, 13, 14, 15, 16, 17, 18, 19, 20, 21, 22, 23, 24, 25, 26, 27, 28, 29, or 30 volumes of the chromatography material is added, while in step (g), a volume of elution buffer corresponding to from about 1 to about 10 volumes, such as from about 2 to about 8 volumes, such as about 5 volumes, or about 1, 2, 3, 4, 5, 6, 7, 8, 9, or 10 volumes, of the chromatography material is added. Similarly, if the method comprises a step (f′), the difference in duration between steps (f)+(f′) compared to step (g) may be accomplished by adding a volume of elution buffer in steps (f)+(f′) which in total is at least 3 times, such as 4 times or more, the volume of elution buffer added in step (g).

The elution buffer applied in steps (b), (f), optionally (f′), and (g) of the above-described method for separating capsids (and as illustrated in FIG. 2) may comprise a salt. The step gradient of increasing conductivity as referred to in step (b) of the method may be a step gradient of increasing salt concentration. In this case, the conductivity-related parameter as referred to in steps (c), (f) and (g) of the method may be the salt concentration. The salt may be a kosmotropic salt, as defined elsewhere herein.

More particularly, the salt may comprise (i) an anion selected from a group consisting of CO₃²⁻, O₄²⁻, S₂O₃²⁻, H₂PO₄⁻, HPO₄²⁻, acetate⁻, citrate⁻, and Cl⁻, and (ii) a cation selected from a group consisting of NH₄⁺, K⁺, Na⁺, and Li⁺. In a currently preferred embodiment, the salt is sodium acetate. However, it is to be understood that other salts consisting of a combination an anion as listed under (i) and a cation as listed under (ii) may alternatively be used to elute the capsids.

Step (b) of the method may comprise adding a volume of the elution buffer corresponding to from about 1 to about 10 volumes, such as from about 2 to about 8 volumes, such as about 5 volumes, or about 1, 2, 3, 4, 5, 6, 7, 8, 9, or 10 volumes, of the chromatography material, per step of the step gradient.

As illustrated in FIG. 3, the present disclosure further provides a method for separating adeno-associated virus capsids fully packaged with genetic material from adeno-associated virus capsids not fully packaged with genetic material, the method comprising:
(I) adding a liquid sample comprising adeno-associated virus capsids of a purity of at least 90% and of a concentration of at least 10¹² adeno-associated virus capsids/ml, of which at least 5% of the adeno-associated virus capsids are adeno-associated virus capsids fully packaged with genetic material, to a strong, or partially strong, anion exchange chromatography material comprising a support and a ligand for binding to the adeno-associated virus capsids, wherein the chromatography material comprises a surface extender connecting the ligand to the support, wherein the surface extender is a polymer, wherein the polymer is selected from:
   (i) a polymer having a naturally occurring skeleton, such as a polysaccharide, such as starch, cellulose, dextran, or agarose, and
   (ii) a polymer having a synthetic skeleton, such as a polyvinyl alcohol, a polyacrylamide, a polymethacrylamide, or a polyvinyl ether;
(II) eluting the adeno-associated virus capsids not fully packaged with genetic material by applying an elution buffer having a pre-determined first value of conductivity or conductivity-related parameter;
(III) eluting the adeno-associated virus capsids fully packaged with genetic material by applying an elution buffer having a pre-determined second value of conductivity or conductivity-related parameter;
the pre-determined first and second value of conductivity or conductivity-related parameter having been determined during separation of a liquid sample comprising adeno-associated virus capsids of a purity of at least 90% and of a concentration of at least 10¹² adeno-associated virus capsids/ml, of which at least 5%, such as 6%, 7%, 8%, 9%, 10%, 20%, 30%, 40%, 50%, 60%, 70%, or 80%, of the adeno-associated virus capsids are adeno-associated virus capsids fully packaged with genetic material, the liquid sample originating from a cell culture harvest from which the liquid sample of step (I) originates;
wherein
   (i) the duration of step (II) is at least 3 times, such as 4 times, 5 times or more, the duration of step (III), and/or
   (ii) the method comprises a step (II′) between step (II) and step (III), wherein step (II′) comprises applying a step elution and/or a gradient of increasing conductivity between the first value and the second value of conductivity or conductivity-related parameter, and the duration of steps (II) and (II′) is at least 3 times, such as 4 times, 5 times or more, the duration of step (III).

The method as illustrated in FIG. 3 is identical to the method as illustrated in FIG. 2 except that the method of FIG. 3 does not comprise steps to determine a first and second value of conductivity or conductivity-related parameter. Instead, the method of FIG. 3 uses a pre-determined first and second value of conductivity or conductivity-related parameter. Optionally, the first and second value of conductivity or conductivity-related parameter may have been pre-determined (i.e., may have been determined previously) by performing steps (a)-(d) of the herein disclosed pre-screening method, as described in detail further above.

It is to be understood that the embodiments and details described above for step (e), (f), optionally (f′), and (g), respectively, of the method of FIG. 2 may equally be applied to step (I), (II), optionally (II′), and (III), respectively, of the method of FIG. 3.

As mentioned above, the chromatography material applied in any of the presently disclosed methods, as described in detail above and as illustrated in FIG. 1, FIG. 2, and FIG. 3, respectively, comprises a strong or partially strong anion exchange chromatography material comprising a support, a ligand for binding to the adeno-associated virus capsids, and a surface extender.

The strong anion exchange chromatography material may be defined by the following Formula I:

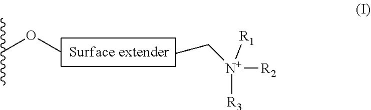

wherein
R₁ is selected from C1-C3 alkyl, and R₂ and R₃ are independently selected from C1-C3 alkyl, CH2OH, and CH2CHOHCH3.

As a non-limiting example, each of R₁, R₂, and R₃ is CH3.

There are currently available chromatography materials comprising a ligand defined by Formula I, wherein each of R₁, R₂, and R₃ is CH3; e.g., a chromatography material made available under the name Capto Q, provided by Cytiva, Sweden (www.cytivalifesciences.com). Capto Q further comprises dextran as surface extender and is a chromatography medium for high-resolution polishing steps in industrial purification processes, e.g., for purification of monoclonal antibodies.

According to another non-limiting example, R₁ and R₂ are ethyl, and R₃ is methyl.

According to yet another non-limiting example, R₁ and R₂ are methyl, and R₃ is CH2CHOHCH3.

The density of ligand defined by Formula I may be from about 60 to about 500 μmol, such as from about 160 to about 350 μmol, such as from about 160 to about 220 μmol, of ligand per ml of the strong anion exchange chromatography material.

Alternatively, the strong, or partially strong, anion exchange chromatography material may be defined by the following Formula II:

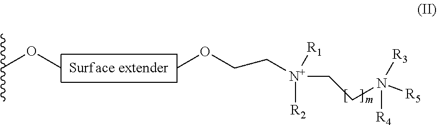

wherein:
m is an integer of from 1 to 3;
R₁ and R₂ are independently selected from a C1-C3 alkyl; R₃, and R₄ are independently selected from C1-C3 alkyl and CH2CHOHCH3; and R₅ is selected from hydrogen, a C1-C3 alkyl and CH2CHOHCH3; provided that if m is 1, the strong, or partially strong, anion exchange chromatography material is defined by the following Formula II:

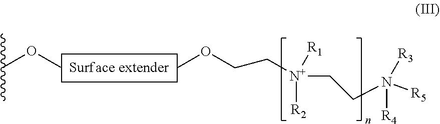

wherein n is an integer of from 0 to 3;
provided that if n is 0, R₃ and R₄ are independently selected from C1-C3 alkyl, and R₅ is hydrogen or CH2CHOHCH3.

As a non-limiting example, the ligand is defined by Formula III and comprises a combination of two or more of the following structures (i)-(iv):
(i) n is 0; R₃ and R₄ are ethyl; and R₅ is hydrogen or CH2CHOHCH3;
(ii) n is 1; R₁, R₂, R₃, R₄ are ethyl; and R₅ is hydrogen or CH2CHOHCH3;
(iii) n is 2; each R₁ and R₂ is ethyl; R₃ and R₄ is ethyl; and R₅ is hydrogen or CH2CHOHCH3;
(iv) n is 3; each R₁ and R₂ is ethyl; R₃ and R₄ is ethyl; and R₅ is hydrogen or CH2CHOHCH3.

One currently available chromatography material comprising a ligand defined by Formula III and comprising a combination of the above-mentioned structures (i)-(iv) is the chromatography resin called Capto DEAE (Cytiva, Sweden). Capto DEAE further comprises dextran as surface extender and is a chromatography medium for high-resolution polishing steps in industrial purification processes, e.g., for purification of monoclonal antibodies.

According to another non-limiting example, the ligand is defined by Formula III, wherein m is 1; n is 1, 2, or 3; each R₁, R₂, R₃, and R₄ is methyl; and R₅ is hydrogen.

According to yet another non-limiting example, the ligand is defined by Formula III, wherein m is 1; n is 1, 2, or 3; each R₁, R₂, R₃, and R₄ is methyl; and R₅ is CH2CHOHCH3.

According to another non-limiting example, the ligand is defined by Formula III, wherein m is 1 and the ligand comprises a combination of two or more of the following structures (i)-(iv):
(i) n is 0; R3 and R4 are methyl; R5 is hydrogen or CH2CHOHCH3;
(ii) n is 1; R1, R2, R3, and R4 are methyl; R5 is hydrogen or CH2CHOHCH3;
(iii) n is 2; each R1 and R2 is methyl; R3 and R4 is methyl; R5 is hydrogen or CH2CHOHCH3;
(iv) n is 3; each R1 and R2 is methyl; R3 and R4 is methyl; R5 is hydrogen or CH2CHOHCH3.

The density of ligand defined by Formula II or Formula III may be from about 60 to about 500 μmol, such as from about 160 to about 350 μmol, such as from about 290 to about 350 μmol, of ligand per ml of the strong anion exchange chromatography material.

As described above, the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose; and
(ii) a polymer having a synthetic skeleton, such as a polyvinyl alcohol, a polyacrylamide, a polymethacrylamide, or a polyvinyl ether.

As a non-limiting example, the surface extender is dextran. The dextran may have a molecular weight of from about 10 to about 2000 kDa, such as about 10, 40, 70, 250, 750, or 2000 kDa, such as 40 kDa. The density of dextran may be from about 5 to about 30 mg dextran per ml of the chromatography material. It is to be understood that the amount of dextran immobilized on the chromatography material may vary, for example depending on the molecular weight of the dextran immobilized. Normally, decreasing amounts are required for increasing molecular weights of dextran.

Steps (a) and (b) of the above-disclosed pre-screening method (FIG. 1) and separation method (FIG. 2), as well as steps (e), (f), (f′), (g) of the separation method of FIG. 2, and steps (I), (II), (II′), and (III) of the separation method of FIG. 3, may comprise applying a buffer having a pH of from about 6.0 to about 10.5, such as from about 7.0 to about 10.0, such as from about 7.5 to about 9.5, or about 6.0, 6.5, 7.0, 7.5, 8.0, 8.5, 9.0, 9.5, 10.0, or 10.5. According to non-limiting examples, as described in Examples 1-3 below, a pH of about 7.0, 9.0, or 9.5 may be applied for a chromatography material comprising a ligand defined by Formula I. Further, as described in Example 1 below, a pH of about 9.0 may be applied for a chromatography material comprising a ligand defined by Formula II or Formula III.

Said buffer is suitably selected from buffers generally recommended for anion exchange chromatography and may for example comprise tris(hydroxymethyl)amino-methane (i.e., Tris), 1,3-bis(tris(hydroxymethyl)methylamino) propane (i.e., bis-Tris propane), triethanolamine, N-methyldiethanolamine, Diethanolamine, 1,3-diaminopropane, or ethanolamine. A person skilled in the art is able to choose a suitable concentration for any one of the above-listed buffers.

In the above-disclosed pre-screening method, step (b) may comprise applying a buffer, optionally one of the buffers mentioned above, wherein the buffer comprises a compound which improves separation between capsids fully packaged with genetic material and capsids not fully packaged with genetic material. If so, in the separation method (FIG. 2), the buffer should comprise the same compound in steps (b), (f), (f′), and (g). Similarly, step (II), (II′), and (III) should comprise applying the same buffer, optionally one of the buffers mentioned above, comprising the same compound which improves separation between fully packaged and not fully packaged capsids, as the buffer applied when pre-determining a first and second value of conductivity or conductivity-related parameter. This compound may or may not be present in a buffer applied in step (a), (e), and (l) of the above-disclosed methods, respectively. Without being bound by theory, such a compound may for example improve separation by influencing interactions between capsid and ligand or interactions between capsid and capsid. Said compound which improves separation may for example be selected from a carbohydrate, a divalent metal ion, and a detergent.

Where said compound which improves separation is a carbohydrate, it may for example be selected from sucrose, sorbitol, and a polysaccharide.

Where said compound which improves separation is a divalent metal ion, it may for example be selected from Mg²⁺, Fe²⁺, and Mn²⁺. The metal ion may be present in the form of a salt, optionally in combination with for example chloride ions or sulphate ions. A non-limiting example of a suitable metal salt to include in the buffer of step (b) is MgCl₂. Non-limiting examples of suitable concentrations of MgCl₂ include from about 0.5 to about 30 mM of MgCl₂, such as from about 1 to about 20 mM, such as from about 2 to about 10 mM, or about 0.5, 1.0, 1.5, 2.0, 2.5, 3, 4, 5, 6, 7, 8, 9, 10, 11, 12, 13, 14, 15, 16, 17, 18, 19, 20, 25, or 30 mM, of MgCl₂.

Where said compound which improves separation is a detergent, it may for example be selected from poloxamer, such as poloxamer 188 or Pluronic™ F68, and polysorbate, such as Tween 20 or Tween 80.

As described in Example 1 below, a non-limiting example of a suitable buffer system to be applied in the above-disclosed methods include a buffer A and a buffer B, both containing 20 mM Bis-Tris Propane (BTP) pH 9.0 and 2 mM MgCl2, and buffer B additionally comprising 250 mM sodium acetate (NaOAc) as elution salt. Buffer A is applied in step (a), (e), and (l), respectively. A step gradient of buffer B is applied in step (b) of the pre-screening method. The first and second value of conductivity or conductivity-related parameter are achieved by applying a mixture of buffer A and buffer B of suitable proportions in steps (f) and (g) of the method of FIG. 2, or in steps (II) and (III) of the method of FIG. 3, respectively.

As described in Example 2 below, another non-limiting example of a suitable buffer system to be applied in the above-disclosed methods include a buffer A and a buffer B, both containing 20 mM Bis-Tris Propane (BTP) pH 7.0, 1% sucrose and 0.1% Pluronic, and buffer B additionally comprising 20 mM MgCl₂.

As described in Example 3 below, another non-limiting example of a suitable buffer system to be applied in the above-disclosed methods include a buffer A and a buffer B, both containing 20 mM Bis-Tris Propane (BTP) pH 7.0 or 9.5 respectively, 18 mM MgCl2, 1% sucrose and 0.1% Pluronic, and buffer B additionally comprising 400 mM NaCl.

The chromatography material applied in the herein disclosed methods comprises a support to which the ligand is coupled. The support may be made from an organic or inorganic material and may be porous or non-porous. In one embodiment, the support is prepared from a native polymer, such as cross-linked carbohydrate material, e.g. agarose, agar, cellulose, dextran, chitosan, konjac, carrageenan, gellan, alginate, pectin, starch, etc. The native polymer supports are easily prepared and optionally cross-linked according to standard methods, such as inverse suspension gelation (S Hjerten: Biochim Biophys Acta 79(2), 393-398 (1964). In an especially advantageous embodiment, the support is a kind of relatively rigid but porous agarose, which is prepared by a method that enhances its flow properties, see e.g. U.S. Pat. No. 6,602,990 (Berg). In an alternative embodiment, the support is prepared from a synthetic polymer or copolymer, such as cross-linked synthetic polymers, e.g. styrene or styrene derivatives, divinylbenzene, acrylamides, acrylate esters, methacrylate esters, vinyl esters, vinyl amides etc. Such synthetic polymers are easily prepared and optionally cross-linked according to standard methods, see e.g. “Styrene based polymer supports developed by suspension polymerization” (R Arshady: Chimica e L'Industria 70(9), 70-75 (1988)). Native or synthetic polymer supports are also available from commercial sources, such as Cytiva, Sweden, for example in the form of porous particles. In yet an alternative embodiment, the support is prepared from an inorganic polymer, such as silica. Inorganic porous and non-porous supports are well known in this field and easily prepared according to standard methods.

The support of the chromatography material may be in the form of particles, such as substantially spherical, elongated or irregularly formed particles.

Where the chromatography material is in the form of particles, the particles may be particles having a homogeneous porosity and being at least partly permeable to adeno-associated virus capsids.

Herein, the term “homogeneous porosity” is intended to mean that a particle having a homogeneous porosity has a homogeneous porosity throughout its entire structure or volume, such that each particle is at least partly permeable to adeno-associated virus capsids throughout its entire structure or volume. In other words, a particle having a homogeneous porosity has a porosity which permits adeno-associated virus capsids to diffuse, completely or at least partly, through its pores, throughout the entire structure or volume of the particle.

Adeno-associated viruses are approx. 20-25 nm in diameter. Since a capsid is the shell of a virus particle, and since adeno-associated viruses do not have a lipoprotein bilayer envelope surrounding the capsid, the size of an adeno-associated virus capsid is approx. 20-25 nm in diameter.

Accordingly, where the chromatography material is in the form of particles having a homogeneous porosity and being at least partly permeable to adeno-associated virus capsids, each particle may suitably comprise pores of a diameter which is >25 nm, i.e., larger than the diameter of the adeno-associated virus capsids to be separated, thereby enabling diffusion of capsids within the entire particle. It is to be understood that for the specific purposes of the present disclosure, i.e., to separate adeno-associated virus capsids, a diameter >25 nm may be of any size >25 nm, including but not limited to 30, 50, 75, 100, 150, or 200 nm.

Further, it is to be understood that a particle having a homogeneous porosity throughout its entire structure or volume nevertheless may comprise pores of different sizes, both pores that are large enough to easily allow capsids to diffuse within the particle and pores that are small enough not to allow diffusion of capsids. This diversity of pore size can be measured by the diffusion coefficient of a molecule of a well-defined molecular weight and hydrodynamic size. As a non-limiting example, dextran, which has a molecular weight of 140-225 kDa or a hydrodynamic diameter of 20-25 nm (i.e., a diameter of the same size as adeno-associated virus capsids), can be used to evaluate the degree of diffusion of adeno-associated virus capsids within the pores of the particles.

The chromatography materials Capto Q and Capto DEAE, advantageously used in Examples 1-3 herein, comprise a support in the form of substantially spherical particles or beads, which have a diameter of approx. 90 μm. This type of particle is a non-limiting example of a particle having a homogeneous porosity (i.e., throughout its entire structure or volume) and being at least partly permeable to adeno-associated virus capsids (i.e., throughout its entire structure or volume).

Suitable particle sizes of a chromatography material for use in the presently disclosed methods may be in a diameter range of 5-500 μm, such as 10-100 μm, e.g., 30-90 μm. In the case of essentially spherical particles, the average particle size may be in the range of 5-1000 μm, such as 10-500. In a specific embodiment, the average particle size is in the range of 10-200 μm. The skilled person in this field can easily choose the suitable particle size and porosity depending on the process to be used. For example, for a large-scale process, for economic reasons, a more porous but rigid support may be preferred to allow processing of large volumes, especially for the capture step. In chromatography, process parameters such as the size and the shape of the column will affect the choice. In an expanded bed process, the matrix commonly contains high density fillers, preferably stainless-steel fillers. For other processes other criteria may affect the nature of the matrix.

The chromatography material may be dried, such as dried particles which upon use are soaked in liquid to retain their original form. For example, such a dried chromatography material may comprise dried agarose particles.

The chromatography material may be in the form of magnetic particles, i.e., magnetic adsorbent beads. The term “magnetic particle” is defined herein as a particle which is able to be attracted by a magnetic field. At the same time, magnetic particles for use in the presently disclosed method shall not aggregate in the absence of a magnetic field. In other words, the magnetic particles shall behave like superparamagnetic particles. The particle may have any symmetric shape, such as a sphere or a cube, or any asymmetric shape. Spherical magnetic particles are often called magnetic beads. It is to be understood that the terms “magnetic particle”, “magnetic bead”, “Mag particle”, “Mag bead”, “magparticle” and “magbead” may be used interchangeably herein, without limiting the scope to magnetic particles having a spherical shape. Separation of biomolecules by use of magnetic adsorbent beads is known in the art. Magnetic particles suitable for use in the presently disclosed method have been described in WO2018122089, which is hereby incorporated by reference in its entirety. A non-limiting example of magnetic particles which may be used in the presently disclosed methods are Mag Sepharose™ PrismA (Cytiva, Sweden).

The support of the chromatography material may alternatively take any other shape conventionally used in separation, such as monoliths, filters or membranes, capillaries, chips, nanofibers, surfaces, etc.

Where the support of the chromatography material comprises a monolith, a suitable pore diameter in the monolith for the purpose of separating adeno-associated virus capsids ranges from a minimum pore diameter of >25 nm, i.e., larger than the diameter of the capsids to be separated, and up to a maximum pore diameter of about 5 m, such as about 0.5, 1.0, 2.0, 3.0, 4.0, or 5.0 m.

Where the support of the chromatography material comprises nanofibers, such nanofibers may for example comprise electrospun polymer nanofibers. When in use, such nanofibers form a stationary phase comprising a plurality of pores through which a mobile phase can permeate.

The support of the chromatography material may comprise a membranous structure, such as a single membrane, a pile of membranes or a filter. The membrane may be an adsorptive membrane. Where the support of the chromatography material comprises a membranous structure, a suitable pore diameter in the membranous structure for the purpose of separating adeno-associated virus capsids ranges from a minimum pore diameter of >25 nm, i.e., larger than the diameter of the capsids to be separated, and up to a maximum pore diameter of about 5 m, such as about 0.5, 1.0, 2.0, 3.0, 4.0, or 5.0 m. Where the chromatography material comprises a membranous structure, such membranous structure may for example comprise a nonwoven web of polymer nanofibers.

Non-limiting examples of suitable polymers may be selected from polysulfones, polyamides, nylon, polyacrylic acid, polymethacrylic acid, polyacrylonitrile, polystyrene, and polyethylene oxide, and mixtures thereof.

Alternatively, the polymer may be a cellulosic polymer, such as selected from a group consisting of cellulose and a partial derivative of cellulose, particularly cellulose ester, cross-linked cellulose, grafted cellulose, or ligand-coupled cellulose. Cellulose fiber chromatography (known as Fibro chromatography; Cytiva, Sweden) is an ultrafast chromatography purification for short process times and high productivity, which utilizes the high flow rates and high capacities of cellulose fiber. Where the support of the chromatography material comprises cellulose fibers such as Fibro, a suitable pore diameter in the cellulose fiber for the purpose of separating adeno-associated virus capsids ranges from a minimum pore diameter of >25 nm, i.e., larger than the diameter of the capsids to be separated, and up to a maximum pore diameter of about 5 μm, such as about 0.1, 0.2, 0.5, 1.0, 2.0, 3.0, 4.0, or 5.0 μm.

The term “membrane chromatography” has its conventional meaning in the field of bioprocessing. In membrane chromatography there is binding of components of a fluid, for example individual molecules, associates or particles, to the surface of a solid phase in contact with the fluid. The active surface of the solid phase is accessible for molecules by convective transport. The advantage of membrane adsorbers over packed chromatography columns is their suitability for being run with much higher flow rates. This is also called convection-based chromatography. A convection-based chromatography matrix includes any matrix in which application of a hydraulic pressure difference between the inflow and outflow of the matrix forces perfusion of the matrix, achieving substantially convective transport of substance(s) into the matrix or out of the matrix, which is effected very rapidly at a high flow rate. Convection-based chromatography and membrane adsorbers are described in for example US20140296464A1, US20160288089A1, WO2018011600A1, WO2018037244A1, WO2013068741A1, WO2015052465A1, U.S. Pat. No. 7,867,784B2, hereby incorporated by reference in their entirety.

In the herein disclosed pre-screening method, methods for separating fully packaged capsids from not fully packaged adeno-associated virus capsids, and uses of chromatography material for separating full from empty capsids, the chromatography material referred to may advantageously be a polishing chromatography material, meaning that the chromatography material is applied in a polishing step.

The term “polishing step” refers in the context of liquid chromatography to a final purification step, wherein trace impurities are removed to leave an active, safe product. Impurities removed during the polishing step are often conformers of the target molecule, i.e., forms of the target molecule having particular molecular conformations, or suspected leakage products. A polishing step may alternatively be called “secondary purification step”.

Further, the liquid sample added in step (a), step (e), and step (I) respectively, of the herein disclosed methods, may advantageously be a pre-purified liquid sample.

The present disclosure further provides a method for separating fully packaged adeno-associated virus capsids from not fully packaged adeno-associated virus capsids, comprising performing the steps (a)-(g) or alternatively steps (I)-(III) as described in detail above, the method further comprising a step (al) which comprises pre-purifying adeno-associated virus capsids by separating adeno-associated virus capsids from an adeno-associated virus capsid-containing cell culture harvest, thereby obtaining a pre-purified liquid sample comprising adeno-associated virus capsids, before adding said pre-purified liquid sample comprising adeno-associated virus capsids to the chromatography material according to step (a), step (e), or step (I), respectively, of the methods described above.

Such a pre-purifying step (al) may alternatively be called a “capture step” and refers in the context of liquid chromatography to the initial step(s) of a separation procedure. Most commonly, a capture step includes clarification (e.g. by filtration, centrifugation, or precipitation), and normally also concentration and/or stabilisation of the sample, and a significant purification from soluble impurities, for example by applying chromatography after the clarification, concentration, and stabilisation of sample. After the capture step, an intermediate purification may follow, which further reduces remaining amounts of impurities such as host cell proteins, DNA, viruses, endotoxins, nutrients, components of a cell culture medium, such as antifoam agents and antibiotics, and product-related impurities, such as aggregates, misfolded species, and aggregates.

Such a pre-purifying step may comprise subjecting the adeno-associated virus capsid-containing cell culture harvest to one or more of the following non-limiting examples of purification methods:
(i) affinity chromatography,
(ii) ion exchange chromatography,
(iii) precipitation or tangential flow filtration (TFF), followed by size-exclusion chromatography, such as by use of for example Capto Core 400 chromatography material (Cytiva, Sweden), which combines flow-through of the capsids with binding of impurities to the chromatography material,
(iv) TFF followed by ion exchange chromatography, and
(v) TFF followed by ion exchange chromatography and Capto Core.

Non-limiting examples of chromatography materials suitable to apply in a pre-purifying step include affinity chromatography material, ion exchange chromatography material, and size-exclusion chromatography material, respectively. The chromatography material may be functionalized with a positively charged group, such as a quaternary amino, quaternary ammonium, or amine group, or a negatively charged group, such as a sulfonate or carboxylate group. The chromatography material may be functionalized with an ion exchanger group, an affinity peptide/protein-based ligand, a hydrophobic interaction ligand, an IMAC ligand, or a DNA based ligand such as Oligo dT.

Herein, the term “cell culture” refers to a culture of cells or a group of cells being cultivated, wherein the cells may be any type of cells, such as bacterial cells, viral cells, fungal cells, insect cells, or mammalian cells. A cell culture may be unclarified, i.e., comprising cells, or may be cell-depleted, i.e., a culture comprising no or few cells but comprising biomolecules released from the cells before removing the cells. Further, an unclarified cell culture may comprise intact cells, disrupted cells, a cell homogenate, and/or a cell lysate.

The term “cell culture harvest” is used herein to denote a cell culture which has been harvested and removed from the vessel or equipment, in which the cells have been cultivated.

The term “separation device” has its conventional meaning in the field of bioprocessing and is to be understood as encompassing any type of separation device which is capable of and suitable for separating and purifying compounds from a fluid containing by-products from the production of the compounds. A separation device may comprise a separation matrix, as further defined elsewhere herein.

Non-limiting examples of separation devices suitable for use in the polishing step according to the presently disclosed method include chromatography columns and membrane devices, as further described elsewhere herein. Such separation devices may suitably comprise chromatography material in the form of a strong anion exchange chromatography material comprising a ligand as defined by Formula I, II or III, as described in detail elsewhere herein.

Non-limiting examples of separation devices suitable for use in a capture step, or pre-purification step, as described herein, are filtration apparatuses, chromatography columns and membrane devices. Chromatography columns suitable for use in the capture step may for example be packed with affinity chromatography material, ion exchange chromatography material, mixed mode chromatography material or hydrophobic interaction chromatography material.

The herein disclosed method for separating fully packaged adeno-associated virus capsids from not fully packaged adeno-associated virus capsids may further comprise subjecting the eluate fractions comprising adeno-associated virus capsids fully packaged with genetic material, eluted in step (g) or step (III), respectively, of the methods as described above, to one or more of the following steps:
h1) concentrating the adeno-associated virus capsids to a pharmaceutically relevant dose,
h2) replacing a buffer applied in step (b)/(g) of the method with a pharmaceutically acceptable buffer, and/or
h3) sterilizing the eluate fractions comprising adeno-associated virus capsids, thereby obtaining a pharmaceutical composition comprising adeno-associated virus capsids.

A person skilled in the art understands that the pharmaceutically relevant dose will depend on various factors such as, but not limited to, the disease or disorder to be treated as well as the weight and condition of the subject to be treated with a pharmaceutical composition.

Pharmaceutically acceptable buffers are well known in the art and can easily be chosen by the skilled person.

For the resulting composition to fulfil all regulatory requirements for pharmaceutical compositions, normally all of the above-listed three steps h1-h3 have to be performed.

In the above-disclosed methods, the adeno-associated virus capsids may advantageously be capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof.

The term “variant” in relation to an adeno-associated virus (AAV) serotype 1, 2, 3, 4, 5, 6, 7, 8, 9, or 10, as listed above, is intended to mean a modified or engineered AAV, in which the capsid structure has been modified to improve clinical performance, for example towards a specific target organ. As a non-limiting example, an AAV8 variant comprises capsid parts of AAV8 and may additionally comprise capsid parts of other AAV serotypes than AAV8, such as AAV5. However, an AAV8 variant as referred to herein must retain a significant structural similarity to a non-modified AAV8 capsid, such as retaining at least 50%, such as 60%, 70%, 80%, or 90%, of the external surface structure of a non-modified AAV8 capsid. This applies equally to a variant of AAV serotype 1, 2, 3, 4, 5, 6, 7, 9, or 10, as compared to a non-modified AAV serotype 1, 2, 3, 4, 5, 6, 7, 9, or 10, respectively. Further, as a non-limiting example, in the context of purification or separation of a variant of AAV8, a “variant” is herein defined as an adeno-associated virus which has a functionally equivalent binding capacity to the ligand of a specified chromatography material, compared to the binding capacity of the original AAV8 to said specified chromatography material. This applies equally to a variant of AAV serotype 1, 2, 3, 4, 5, 6, 7, 9, or 10, as compared to the original AAV serotype 1, 2, 3, 4, 5, 6, 7, 9, or 10, respectively. The specified chromatography material may, for example, be a strong anion exchange chromatography material as disclosed in more detail elsewhere herein. A variant of an adeno-associated virus may for example be obtained by spontaneous mutation, or by engineered modification (i.e., obtained by human interaction), of one or more nucleotides of the genome of the adeno-associated virus.

According to a currently preferred embodiment, in the separation method as illustrated in FIG. 2, the chromatography material is defined by Formula IV:

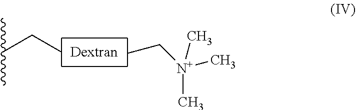

wherein the elution buffer of steps (b), (f), optionally (f′), and (g) comprises sodium acetate. Said method may be applied for separation of AAV capsids of any serotype or variant as described above. In particular, the capsids to be separated may be capsids of the AAV9 serotype or a variant thereof.

According to another currently preferred embodiment, in the separation method as illustrated in FIG. 3, the chromatography material is defined by Formula IV:

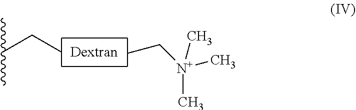

wherein the elution buffer of steps (II), optionally (II′), and (III) comprises sodium acetate. Said method may be applied for separation of AAV capsids of any serotype or variant as described above. In particular, the capsids to be separated may be capsids of the AAV9 serotype or a variant thereof.

The present disclosure further provides use of an anion exchange chromatography material comprising a support, a ligand, and a surface extender connecting the ligand to the support, and being defined by Formula IV:

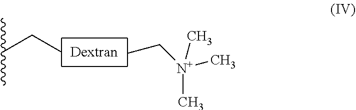

for separating adeno-associated virus capsids fully packaged with genetic material from adeno-associated virus capsids not fully packaged with genetic material, comprising determining elution conditions suitable for separating adeno-associated virus capsids
fully packaged with genetic material from adeno-associated virus capsids not fully packaged with genetic material,
wherein said elution conditions are determined by performing the steps:
a. adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, to the chromatography material;
b. eluting the adeno-associated virus capsids not fully packaged with genetic material and the adeno-associated virus capsids fully packaged with genetic material from the chromatography material by applying an elution buffer comprising a step gradient of increasing conductivity, which starts at from about 0 to about 5 mS/cm, and which increases by from about 0.5 to about 3 mS/cm per step, such as from about 1 to about 2 mS/cm per step, such as from about 1.2 to about 1.5 mS/cm per step, at least up to and including a conductivity at which the adeno-associated virus capsids not fully packaged with genetic material and the adeno-associated virus capsids fully packaged with genetic material have been eluted from the chromatography material;
c. based on an elution profile obtained in step (b), determining a first value of conductivity or conductivity-related parameter, which is suitable for eluting the adeno-associated virus capsids not fully packaged with genetic material, and
d. based on the elution profile obtained in step (b), determining a second value of conductivity or conductivity-related parameter, which is suitable for eluting the adeno-associated virus capsids fully packaged with genetic material.

In the herein disclosed use, the adeno-associated virus capsids fully packaged with genetic material may be separated from adeno-associated virus capsids not fully packaged with genetic material by performing steps (a)-(d) as described above, and further by performing the steps:
e. adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, the liquid sample originating from a cell culture harvest from which the liquid sample of step (a) originated, to the chromatography material;
f. eluting the adeno-associated virus capsids not fully packaged with genetic material by applying an elution buffer having the first value of conductivity or conductivity-related parameter as determined in step (c); and
g. eluting the adeno-associated virus capsids fully packaged with genetic material by applying an elution buffer having the second value of conductivity or conductivity-related parameter as determined in step (d);
   wherein
   (i) the duration of step (f) is at least 3 times, such as 4 times, the duration of step (g), and/or
   (ii) the method comprises a step (f′) between step (f) and step (g), wherein step (f′) comprises applying an additional step elution and/or a gradient of increasing conductivity between the first value and the second value of conductivity or conductivity-related parameter, and the duration of steps (f) and (f′) is at least 3 times, such as 4 times, the duration of step (g).

Preferably, the elution buffer of step (b) of the above-described use, and where applicable also the elution buffer of steps (f) and (g) of the above-described use, comprises sodium acetate. Said use may be applied to separation of AAV capsids on any serotype or variant as described above. In particular, the capsids to be separated may be capsids of the AAV9 serotype or a variant thereof.

Devices or compositions “comprising” one or more recited components may also include other components not specifically recited. The term “comprising” includes as a subset “consisting essentially of” which means that the device or composition has the components listed without other features or components being present. Likewise, methods “comprising” one or more recited steps may also include other steps not specifically recited.

The singular “a” and “an” shall be construed as including also the plural.

### EXAMPLES

### Example 1: Separation of Capsids of Serotypes AAV2, a AV5, AAV8 and AAV9 on Anion Exchange Chromatography Materials Using Increasing Sodium Acetate Step Gradient for Elution

### Chromatography Materials

The following currently available anion exchange chromatography materials gave improved results, as described further below:

### Capto Q (Cytiva, Sweden):

Ligand: Quaternary amine
Particle size, d_{50V}: ˜90 μm
Matrix: Highly cross-linked agarose with dextran surface extender
Ionic Capacity: 0.16-0.22 mmol Cl⁻/ml medium
pH stability, operational: 2-12.

### Capto DEAE (Cytiva, Sweden):

Ligand: Diethylaminoethyl, partially quaternized amine groups
Particle size, d_{50V}: ˜90 μm
Matrix: Highly cross-linked agarose with dextran surface extender
Ionic Capacity: 0.29-0.35 mmol Cl⁻/ml medium
pH stability, operational: 2-9.

Further, the currently available Capto Q ImpRes (Cytiva, Sweden) was also tested as described below. The support material of the ImpRes resin consists of substantially spherical particles or beads, which have a diameter of 40 μm.

### Equipment and Samples

Each resin was packed in a Tricorn 5 column (2 mL) according to the packing instructions. The runs were performed using an Akta Pure P25 system (P25-20031) with a flowrate of 1 CV/min (i.e., 2 mL/min), with the mixer of the system disconnected in order to minimize the dead volume and to get sharp conductivity steps. The sample was applied to the previously equilibrated column using a capillary loop. Typically, samples applied to each resin comprised affinity purified, or affinity and size exclusion purified, AAV2, AAV5, AAV8 or AAV9, respectively, at a concentration of approx. 5×10¹² AAV capsids, containing a mixture of full and empty capsids (>5% full capsids, as follows: AAV2 7-10%, AAV5 47%, AAV8 11-35%, AAV9 40%). The material needs to have low conductivity (1-3 mS/cm) to ensure binding of AAV to the anion exchange ligand.

The 280 and 260 nm UV absorbance were monitored during the runs and the 260/280 ratios were used as a diagnostic tool to navigate in the chromatogram and distinguish between full and empty capsid populations. The chromatograms were analyzed using the Evaluation package of Unicorn. A 260/280 ratio above 1.2 is considered to indicate 100% full capsids, and a 260/280 ratio of approx. or below 0.6-0.7 is considered to indicate 100% empty capsids. Blank runs with the buffers without AAV were performed to subtract any background signal if needed, to ensure removal of potential UV signals from the buffers.

### Process Conditions and Results

Currently available cation exchange resin, Capto S, and prototype cation exchange resin, Capto CM Dx ImpRes, were evaluated with acetate buffer at pH 4.5 and 5, with and without additives (0.1% poloxamer 188, 1% sucrose, with different elution salts (NaCl, NaOAc, NH₄Cl or NH₄SO₄ up to 500 mM) and additive salts (MgCl₂ and MgSO4 up to 20 mM), by applying an isocratic elution or continuous gradient elution, respectively. None of the above-mentioned conditions or resins resulted in a good baseline separation of full and empty capsids (data not shown).

Each of the currently available anion exchange resins with dextran extenders, Capto DEAE (partially strong anion exchange) and Capto Q (strong anion exchange), were evaluated using a buffer system including a buffer A and a buffer B, both containing 20 mM Bis-Tris Propane (BTP) pH 9.0 and 2 mM MgCl₂, and buffer B additionally comprising 250 mM sodium acetate (NaOAc) as elution salt.

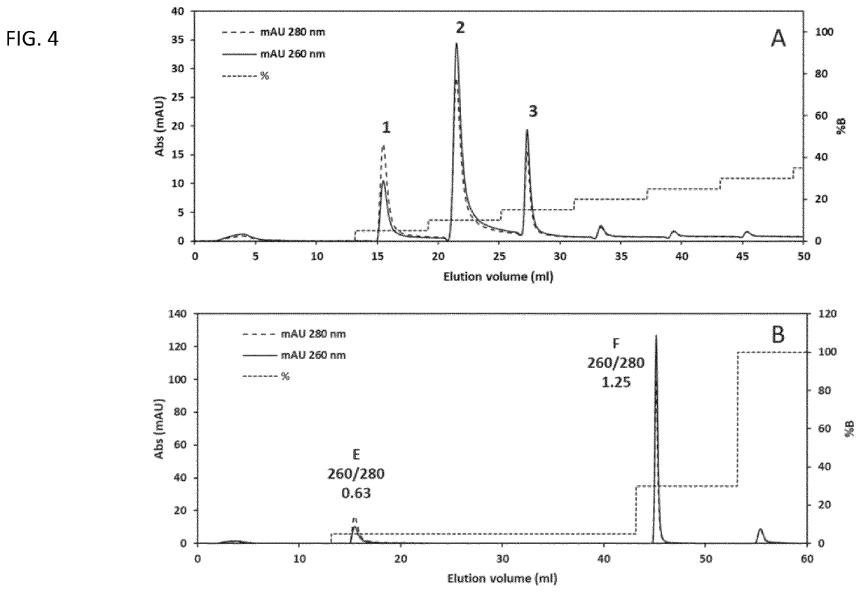

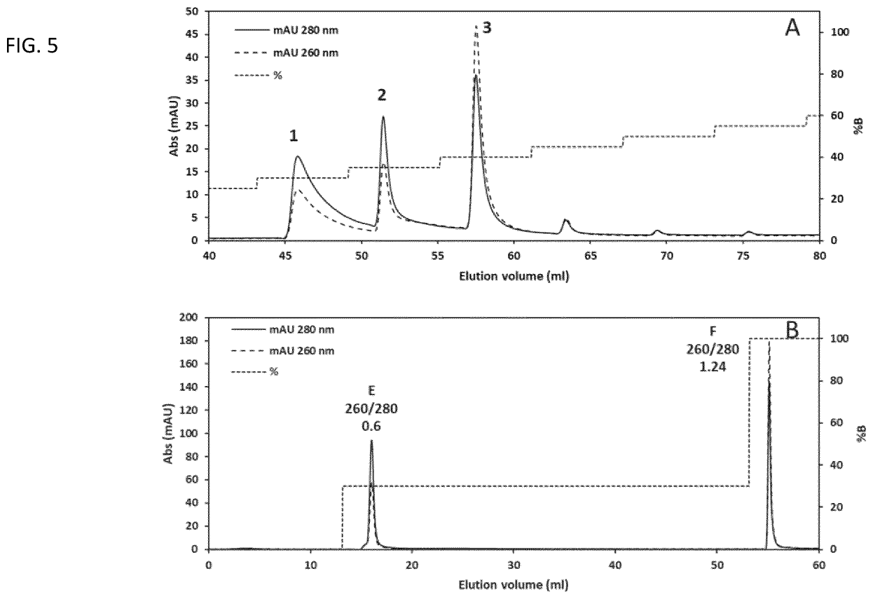

In FIGS. 4-7, the y-axis on the right-hand side of each chromatogram denotes the percentage of buffer B included in the resulting elution buffer (the rest being buffer A) during elution from the chromatography material.

### Pre-Screening Method

The step gradient applied in the pre-screening method comprised short steps (3 CV) of increasing concentration of NaOAc, more particularly a 12.5 mM increase per step, i.e., an increase in concentration of 5% per step of the 250 mM NaOAc of buffer B, which corresponds to an increase in conductivity of approx. 1.2-1.5 mS/cm per step.
Equilibration: 5 CV buffer A
Injection: empty loop/w 3 ml buffer A
Wash: 5 CV buffer A
Step gradient: 5% increasing steps of buffer B, 3 CV each
Wash: 100% buffer B, 5 CV
Re-equilibration: 5 CV buffer A

### Capto Q

FIG. 4A shows the results of the pre-screening method for AAV9 separated on Capto Q resin. Multiple peaks close to each other can be seen in the chromatogram (peaks no. 1, 2, and 3 of FIG. 4A). Peaks no. 1, 2, and 3 of FIG. 4A contained different ratios of full and empty capsids. Peak no. 1 contained mainly empty capsids, while peak no. 2 and 3 mainly contained full capsids. The UV260/280 ratios were as follows: 0.62 for peak 1, 1.21 for peak 2, and 1.26 for peak 3.

Based on the chromatogram of FIG. 4A, a first value of conductivity was determined as being suitable for elution of empty capsids. The first value of conductivity determined corresponded to the concentration of NaOAc applied when eluting the first peak containing empty and/or full capsids, i.e., peak no. 1. The first value of conductivity chosen was approx. 1.2-1.5 mS/cm, corresponding to 5% of buffer B, i.e., 12.5 mM NaOAc.

Also based on the chromatogram of FIG. 4A, a second value of conductivity was determined as being suitable for elution of full capsids. The second value of conductivity chosen corresponded to a concentration of NaOAc higher than the concentration of NaOAc applied when eluting the last peak containing empty and/or full capsids, i.e., peak no. 3. The second value of conductivity chosen was approx. 7.2-9.0 mS/cm, corresponding to 30% of buffer B, i.e., 75 mM NaOAc.

Here, it is noted that the second value of conductivity could instead have been chosen so as to correspond to the concentration of NaOAc applied when eluting the last peak containing empty and/or full capsids or any higher value. Thus, the second value of conductivity could have been approx. 3.6-4.5 mS/cm, corresponding to 15% of buffer B, i.e., 37.5 mM NaOAc as applied when eluting peak no. 3, or any higher value, i.e., ≥3.6 mS/cm, corresponding to ≥37.5 mM NaOAc.

FIG. 5A shows the results of the pre-screening method for AAV8 separated on Capto Q resin. Multiple peaks close to each other can be seen in the chromatogram (peaks no. 1, 2, and 3 of FIG. 5A). Peaks no. 1, 2, and 3 of FIG. 5A contained different ratios of full and empty capsids. Peaks no. 1 and 2 contained mainly empty capsids, while peak no. 3 mainly contained full capsids. The UV260/280 ratios were as follows: 0.6 for peak 1, 0.66 for peak 2, and 1.3 for peak 3.

Based on the chromatogram of FIG. 5A, a first value of conductivity was determined as being suitable for elution of empty capsids. The first value of conductivity determined corresponded to the concentration of NaOAc applied when eluting the first peak containing empty and/or full capsids, i.e., peak no. 1. The first value of conductivity chosen was approx. 7.2-9.0 mS/cm, corresponding to 30% of buffer B, i.e., 75 mM NaOAc.

Also based on the chromatogram of FIG. 5A, a second value of conductivity was determined as being suitable for elution of full capsids. The second value of conductivity chosen corresponded to a concentration of NaOAc higher than the concentration of NaOAc applied when eluting the last peak containing empty and/or full capsids, i.e., peak no. 3. The second value of conductivity chosen was approx. 24-30 mS/cm, corresponding to 100% of buffer B, i.e., 250 mM NaOAc.

Here, it is noted that the second value of conductivity could instead have been chosen so as to correspond to the concentration of NaOAc applied when eluting the last peak containing empty and/or full capsids or any higher value. Thus, the second value of conductivity could have been approx. 9.6-12.0 mS/cm, corresponding to 40% of buffer B, i.e., 100 mM NaOAc as applied when eluting peak no. 3, or any higher value, i.e., >9.6 mS/cm, corresponding to ≥100 mM NaOAc.

### Capto DEAE

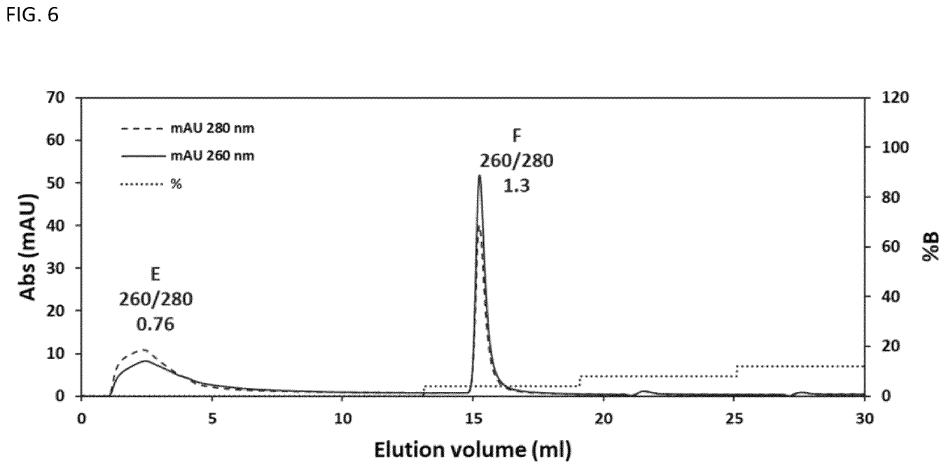

FIG. 6 shows the results of a pre-screening method for elution of AAV9 on Capto DEAE resin. Using the same conditions as described above for Capto Q (buffer A: 20 mM BTP pH 9.0, 2 mM MgCl₂; buffer B: buffer A +250 mM NaOAc) but using 4% incremental elution steps of buffer B (3 CV each) in the pre-screening method, the AAV9 empty capsids eluted in flow-through and the AAV9 full capsids eluted in the first step of the step gradient. The UV 260:280 ratio was 0.76 in the first peak (i.e., the flow through peak), suggesting mainly empty AAV9 capsids but also a small amount of full capsids. The UV260:280 ratio was 1.3 in the second peak, indicating high purity of AAV9 full capsids (FIG. 6).

Based on the chromatogram of FIG. 6, the first value of conductivity suitable for elution of AAV9 empty capsids was determined to be approx. 0 mS/cm, corresponding to 0% of buffer B, and the second value of conductivity suitable for elution of AAV9 full capsids was determined to be approx. 1 mS/cm, corresponding to 4% of buffer B, i.e., 10 mM NaOAc.

### Two-Step Elution Method

The first and second value of conductivity as determined in the above-described pre-screening method were applied in a subsequent two-step elution method for separating full capsids from empty capsids.
Equilibration: 5 CV buffer A
Injection: empty loop/w 3 ml buffer A
Wash: 5 CV buffer A
Step elution: step 1, first value of conductivity, 20 CV; step 2, second value of conductivity, 5 CV
Re-equilibration: 5 CV buffer A

### Capto Q

When using the Capto Q resin, all tested serotypes (AAV2, AAV5, AAV8, and AAV9) resulted in a good separation of full and empty capsids.

FIG. 4B shows the results of the two-step elution method for AAV9 on Capto Q resin, applying a first value of conductivity corresponding to 5% buffer B in the first step and a second value of conductivity corresponding to 30% buffer B in the second step, as explained above. Full (F) and empty (E) capsids, and UV260:280 ratios are indicated in FIG. 4B.

FIG. 5B shows the results of the two-step elution method for AAV8 on Capto Q resin, applying 30% buffer B in the first step and 100% buffer B in the second step, as explained above. Full (F) and empty (E) capsids, and UV260:280 ratios are indicated in FIG. 5B.

In the AAV9 sample tested, the peaks containing empty and/or full capsids were also analyzed with qPCR, showing very small amounts (0.3%) of full capsids in the empty peak and an overall 91% viral genome recovery in the full peak.

### Capto DEAE

AAV9 capsids were separated on the Capto DEAE resin by applying the conditions established in the pre-screening method described above, i.e., a first value of conductivity corresponding to 0% of buffer B and a second value of conductivity corresponding to 4% of buffer B. The resulting separation of full and empty capsids was as shown in FIG. 6 (described above).

Capto Q ImpRes (without Dextran Extenders)

Capto Q ImpRes resin was evaluated for separation of AAV9 and AAV5, respectively, under conditions identical to those described above, except that a flowrate of 1 ml/min was applied due to high delta column pressures. The resin did not work for AAV5 but worked adequately for pre-screening and 2-step elution for separation of AAV9 full capsids from AAV9 empty capsids (results not shown). However, Capto Q ImpRes (without extenders) does not bind AAV9 empty capsids (which thereby elute in the flow-through) and only binds AAV9 full capsids weakly, and thus provides a less robust separation method than Capto Q (with extenders).

### Example 2: Separation of Capsids of Serotypes AAV5 and AAV9 on Anion Exchange Chromatography Material Using Increasing Magnesium Chloride Step Gradient for Elution

The Capto Q resin was evaluated for separation of AAV9 and AAV5, respectively, by applying a pre-screening method followed by a 2-step elution as described in Example 1, with the difference that buffer A and buffer B of the buffer system both included 20 mM Bis-Tris Propane (BTP) pH 7.0 (AAV5) or pH 9.5 (AAV9), 1% sucrose and 0.1% Pluronic, and buffer B additionally comprising 20 mM MgCl₂.

### Pre-Screening Method

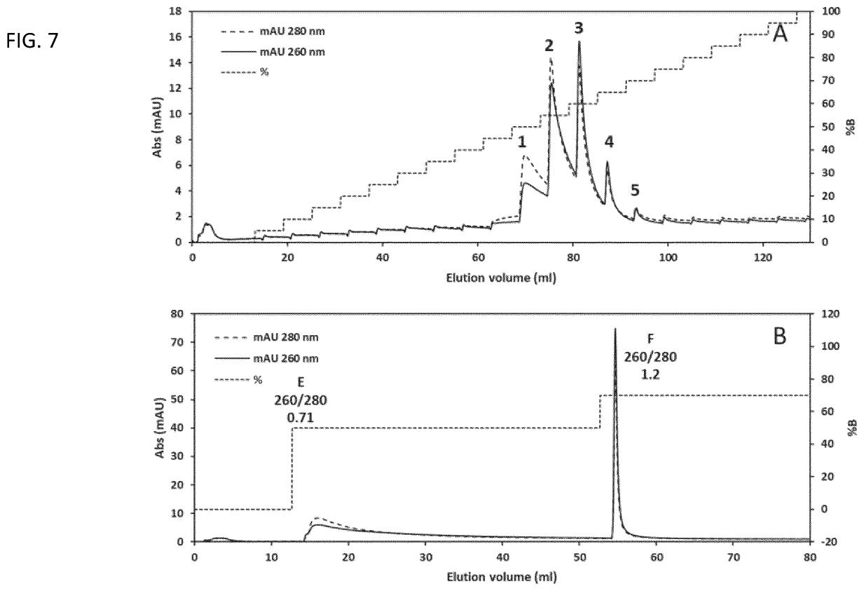

Equilibration: 5 CV buffer A
Injection: empty loop/w 3 ml buffer A
Wash: 5 CV buffer A
Step gradient elution: 5% increasing steps of buffer B, 3 CV each
Wash: 5 CV 100% buffer B
Re-equilibration: 5 CV buffer A
   FIG. 7A shows the results of the pre-screening method for AAV5 separated on Capto Q resin. Multiple peaks close to each other can be seen in the chromatogram (peaks no. 1, 2, 3 and 4 of FIG. 7A). Peaks no. 1, 2, 3, 4, and 5 contained different ratios of full and empty capsids. Peaks no. 1 and 2 contained mainly empty capsids, while peaks no. 3, 4, and 5 mainly contained full capsids. The UV260:280 ratios were as follows: 0.68 for peak 1, 0.87 for peak 2, 1.14 for peak 3, 1.1 for peak 4, and 1 for peak 5.

Based on the chromatogram of FIG. 7A, a first value of conductivity was determined as being suitable for elution of empty capsids. The first value of conductivity determined corresponded to the concentration of MgCl₂ applied when eluting the first peak containing empty and/or full capsids, i.e., peak no. 1. The first value of conductivity chosen was approx. 4.6 mS/cm, corresponding to 50% of buffer B, i.e., 10 mM MgCl₂.

Further, based on the chromatogram of FIG. 7A, a second value of conductivity was determined as being suitable for elution of full capsids. The second value of conductivity chosen corresponded to a concentration of MgCl₂ identical to the concentration of MgCl₂ applied when eluting the last peak containing empty and/or full capsids, i.e., peak no. 5. The second value of conductivity chosen was approx 5.3 mS/cm, corresponding to 70% of buffer B, i.e., 14 mM MgCl₂.

Alternatively, the second value of conductivity could have been chosen to be higher than the concentration of MgCl₂ applied when eluting the last peak containing empty and/or full capsids, i.e., peak no. 5.

### Two-Step Elution Method

The first and second value of conductivity as determined in the above-described pre-screening method were applied in a subsequent two-step elution method for separating full capsids from empty capsids.
Equilibration: 5 CV buffer A
Injection: empty loop/w 3 ml buffer A
Wash: 5 CV buffer A
Step elution: step 1, 50% buffer B, 20 CV; step 2, 70% buffer B, 20 CV
Re-equilibration: 5 CV buffer A

FIG. 7B shows the results of the two-step elution method for AAV5 on Capto Q resin, applying 50% buffer B in the first step and 70% buffer B in the second step, as explained above. Full (F) and empty (E) capsids, and UV260:280 ratios are indicated in FIG. 7B. Here, considering that the second peak (full capsids) is very sharp (hardly any tailing) it is to be noted that the two-step elution method would have worked equally well by applying a shorter duration of step 2, corresponding to 5 CV (as in the previous examples).

### Alternative Buffer System

An alternative buffer system was also tested, including buffer A and buffer B having a higher pH, both containing 20 mM Bis-Tris Propane (BTP) pH 9.5, 1% sucrose and 0.1% Pluronic, and buffer B additionally comprising 30 mM MgCl₂, i.e., a higher concentration of MgCl₂. Here, in the pre-screening method, 3.5% steps of increasing MgCl₂ were applied. The first and second conductivity values determined corresponded to 68% and 90% of buffer B, respectively. Said conductivity values were then applied in the two-step elution method.

The above-described conditions resulted in successful baseline separation of AAV5 (results for the alternative buffer system not shown). AAV9 binds less strongly to anion exchange, however conditions involving higher pH (such as 9.5), and application of small conductivity increase elution steps resulted in acceptable performance (results not shown).

### Example 3: Separation of AAV9 Capsids Under Variable Conditions

Experimental designs for separation of fully packaged AAV9 capsids from empty and partially packaged AAV9 capsids are performed with equipment and samples as in Example 1 and Example 2 above, and further by use of anion exchange chromatography material as in Example 1 and Example 2, with the following variations:

### In Terms of Capsids:

1. Engineered capsid variants based on AAV9
2. AAV9 capsids with other and longer insert than the insert included in the capsids of Example 1 and Example 2, which was Green Fluorescent Protein (GFP).

### In Terms of Surface Extenders:

1) Different size (kDa) of dextran (T10, T70, T250);
2) Different amounts of dextran;
3) Use of alternatives to dextran, e.g., poly alcohol based on glycidol.

### In Terms of Ligand Density:

1) Capto Q: 60-160, 220-260 μmol/mL;
2) Capto DEAE: 150-290, 350-400 μmol/mL.

### In Terms of Chromatography Material Support:

1) Smaller resin bead size: 35-90 μm;
2) Resin beads with larger pore sizes than Capto Q and Capto DEAE;
3) Magnetic beads in batch mode.

### In Terms of Ligand Chemistry:

1) Capto DEAE ligand with different levels of quaternization (ligand according to Formula III); 2) Capto Q analogues (ligand according to Formula I):
   a. R1, R2 is ethyl; R3 is methyl;
   b. R1, R2 is methyl; R3 is CH2CHOHCH3;
3) Capto DEAE analogues (ligand according to Formula II):
   a. m=1; n=1; R1, R2, R3, and R4=methyl; R5=H;
   b. m=1; n=1; R1, R2, R3, and R4=methyl; R5=CH2CHOHCH3.
4) Capto DEAE analogues (ligand according to Formula III):
   a. n=1; R1, R2, R3, and R4=methyl; R5=H or not present.

### In Terms of Buffers and Elution Conditions:

1) Different concentrations of MgCl₂ between 1-20 mM;
2) Different kosmotropic salts for elution, step gradient 0.1-1M, with or without MgCl₂ as in 1);
3) Different pH step gradient pH 4-10, with or without MgCl₂ as in 1);
4) Different buffers:
   a. Tris
   b. N-Methyldiethanolamine
5) Linear gradient elutions with pH, kosmotropic salt for elution, MgCl₂ as in 1), 2), 3) and 4);
6) Additional steps, e.g., a third elution step or a linear elution gradient between the two elution steps in the 2-step elution protocol according to Example 1;
7) Conditions suitable for flow-through separation, where one analyte binds to the chromatography material while another analyte does not bind to it (anion exchange resins bind more strongly to full capsids than to empty capsids; thus empty capsids may elute with the flow-through);
8) All of the above with or without additives like sucrose (0.1-5%) and poloxamer 188 detergent (0.01-1%).

### Example 4: Separation of Capsids of Different Adeno-Associated Virus Serotypes Under Variable Conditions

Experimental designs for separation of full capsids from empty capsids of adeno-associated virus serotypes AAV1, AAV2, AAV3, AAV4, AAV5, AAV6, AAV7, AAV8, and AAV10, and variants thereof, are performed according to the variable conditions of Examples 1-4 above.

It is to be understood that the present disclosure is not restricted to the above-described exemplifying embodiments thereof and that several conceivable modifications of the present disclosure are possible within the scope of the following claims.

### REFERENCES

Weihong Qu et al, Scalable Downstream Strategies for Purification of Recombinant Adeno-Associated Virus Vectors in Light of the Properties, Current Pharmaceutical Biotechnology 2015 August; 16(8): 684-695
Hejmowski Adam L et al, Novel anion exchange membrane chromatography method for the separation of empty and full adeno-associated virus, Biotechnol. J. 2021; 2100219. https://doi.org/10.1002/biot.202100219
Xiaotong Fu et al, Analytical Strategies for Quantification of Adeno-Associated Virus Empty Capsids to Support Process Development, Human gene therapy methods, 2019, 30(4): 144-152
Moelbert Susanne et al, Kosmotropes and chaotropes: modelling preferential exclusion, binding and aggregate stability, Biophysical Chemistry, 2004 December, 112(1): 45-57
Hyde A M et al, General Principles and Strategies for Salting-Out Informed by the Hofmeister Series, Organic Process Research & Development, 2017, 21 (9): 1355-1370.

1. A method for determining elution conditions suitable for separating adeno-associated virus capsids fully packaged with genetic material from adeno-associated virus capsids not fully packaged with genetic material, the method comprising the following steps:
a. adding a liquid sample comprising adeno-associated virus capsids of a purity of at least 90% and of a concentration of at least 10¹² adeno-associated virus capsids/ml, of which at least 5% of the adeno-associated virus capsids are adeno-associated virus capsids fully packaged with genetic material, to a strong, or partially strong, anion exchange chromatography material comprising a support and a ligand for binding to the adeno-associated virus capsids, wherein the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose, and
(ii) a polymer having a synthetic skeleton, such as a polyvinyl alcohol, a polyacrylamide, a polymethacrylamide, or a polyvinyl ether;
b. eluting the adeno-associated virus capsids not fully packaged with genetic material and the adeno-associated virus capsids fully packaged with genetic material from the chromatography material by applying an elution buffer comprising a step gradient of increasing conductivity, which starts at from about 0 to about 5 mS/cm, and which increases by from about 0.5 to about 3 mS/cm per step, at least up to and including a conductivity at which the adeno-associated virus capsids not fully packaged with genetic material and the adeno-associated virus capsids fully packaged with genetic material have been eluted from the chromatography material;
c. based on an elution profile obtained in step (b), determining a first value of conductivity or conductivity-related parameter, which is suitable for eluting the adeno-associated virus capsids not fully packaged with genetic material, and
d. based on the elution profile obtained in step (b), determining a second value of conductivity or conductivity-related parameter, which is suitable for eluting the adeno-associated virus capsids fully packaged with genetic material.

2. The method of claim 1, wherein the step gradient of increasing conductivity in step (b) is a step gradient of increasing salt concentration, optionally wherein the conductivity-related parameter in steps (c) and (d) is the salt concentration.

3. The method of claim 2, wherein the salt is a kosmotropic salt.

4. The method of claim 2 or 3, wherein the salt comprises (i) an anion selected from a group consisting of CO₃²⁻, SO₄²⁻, S₂O₃²⁻, H₂PO₄⁻, HPO₄²⁻, acetate⁻, citrate⁻, and Cl⁻, and (ii) a cation selected from a group consisting of NH₄⁺, K⁺, Na⁺, and Li⁺; optionally wherein the salt is sodium acetate.

5. The method of any preceding claim, wherein step (b) comprises adding a volume of the elution buffer corresponding to from about 1 to about 10 volumes of the chromatography material, per step of the step gradient.

6. A method for separating adeno-associated virus capsids fully packaged with genetic material from adeno-associated virus capsids not fully packaged with genetic material, the method comprising steps (a)-(d) of claim 1, and further comprising the steps:
e. adding a liquid sample comprising adeno-associated virus capsids of a purity of at least 90% and of a concentration of at least 10¹² adeno-associated virus capsids/ml, of which at least 5% of the adeno-associated virus capsids are adeno-associated virus capsids fully packaged with genetic material, the liquid sample originating from a cell culture harvest from which the liquid sample of step (a) originated, to the chromatography material as defined in step (a);
f. eluting the adeno-associated virus capsids not fully packaged with genetic material by applying an elution buffer having the first value of conductivity or conductivity-related parameter as determined in step (c); and
g. eluting the adeno-associated virus capsids fully packaged with genetic material by applying an elution buffer having the second value of conductivity or conductivity-related parameter as determined in step (d);
wherein
(i) the duration of step (f) is at least 3 times the duration of step (g), and/or
(ii) the method comprises a step (f′) between step (f) and step (g), wherein step (f′) comprises applying an additional step elution and/or a gradient of increasing conductivity between the first value and the second value of conductivity or conductivity-related parameter, and the duration of steps (f) and (f′) is at least 3 times the duration of step (g).

7. The method of claim 6, wherein the elution buffer comprises a salt and the step gradient of increasing conductivity in step (b) is a step gradient of increasing salt concentration, optionally wherein the conductivity-related parameter in steps (c), (f) and (g) is the salt concentration.

8. The method of claim 7, wherein the salt is a kosmotropic salt.

9. The method of claim 7 or 8, wherein the salt comprises (i) an anion selected from a group consisting of CO₃²⁻, SO₄²⁻, S₂O₃²⁻, H₂PO₄⁻, HPO₄²⁻, acetate⁻, citrate⁻, and Cl⁻, and (ii) a cation selected from a group consisting of NH₄⁺, K⁺, Na⁺, and Li⁺; optionally wherein the salt is sodium acetate.

10. The method of any one of claims 6-9, wherein step (b) comprises adding a volume of the elution buffer corresponding to from about 1 to about 10 volumes of the chromatography material, per step of the step gradient.

11. A method for separating adeno-associated virus capsids fully packaged with genetic material from adeno-associated virus capsids not fully packaged with genetic material, the method comprising:
(I) adding a liquid sample comprising adeno-associated virus capsids of a purity of at least 90% and of a concentration of at least 10¹² adeno-associated virus capsids/ml, of which at least 5% of the adeno-associated virus capsids are adeno-associated virus capsids fully packaged with genetic material, to a strong, or partially strong, anion exchange chromatography material comprising a support and a ligand for binding to the adeno-associated virus capsids,
wherein the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose, and
(ii) a polymer having a synthetic skeleton, such as a polyvinyl alcohol, a polyacrylamide, a polymethacrylamide, or a polyvinyl ether;
(II) eluting the adeno-associated virus capsids not fully packaged with genetic material by applying an elution buffer having a pre-determined first value of conductivity or conductivity-related parameter;
(III) eluting the adeno-associated virus capsids fully packaged with genetic material by applying an elution buffer having a pre-determined second value of conductivity or conductivity-related parameter;
the pre-determined first and second value of conductivity or conductivity-related parameter having been determined during separation of a liquid sample comprising adeno-associated virus capsids of a purity of at least 90% and of a concentration of at least 10¹² adeno-associated virus capsids/ml, of which at least 5% of the adeno-associated virus capsids are adeno-associated virus capsids fully packaged with genetic material, the liquid sample originating from a cell culture harvest from which the liquid sample of step (I) originates, optionally having been determined by performing the method of steps (a)-(d) of claim 1;
wherein
(i) the duration of step (II) is at least 3 times the duration of step (III), and/or
(ii) the method comprises a step (II′) between step (II) and step (III), wherein step (II′) comprises applying a step elution and/or a gradient of increasing conductivity between the first value and the second value of conductivity or conductivity-related parameter, and the duration of steps (II) and (II′) is at least 3 times the duration of step (III).

12. The method of any preceding claim, wherein the chromatography material is defined by:
i) Formula I: 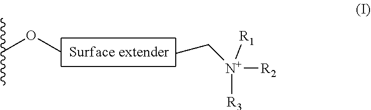
wherein
R₁ is selected from C₁-C₃ alkyl, and R₂ and R₃ are independently selected from C1-C3 alkyl, CH2OH, and CH2CHOHCH3;
optionally wherein each of R₁, R₂, and R₃ is CH3;
or
ii) Formula II: 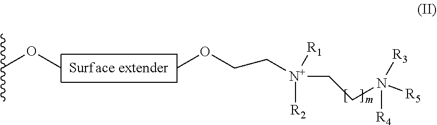
wherein:
m is an integer of from 1 to 3;
R₁ and R₂ are independently selected from a C1-C3 alkyl; R₃, and R₄ are independently selected from C1-C3 alkyl and CH2CHOHCH3; and R₅ is selected from hydrogen, a C1-C3 alkyl and CH2CHOHCH3;
provided that if m is 1, the chromatography material is defined by Formula III: 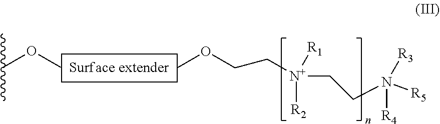
wherein n is an integer of from 0 to 3;
provided that if n is 0, R₃ and R₄ are independently selected from C1-C3 alkyl, and R₅ is hydrogen or CH2CHOHCH3;
optionally wherein the chromatography material is defined by Formula III and comprises a combination of two or more of the following structures (i)-(iv):
(i) n is 0; R₃ and R₄ are ethyl; and R₅ is hydrogen or CH2CHOHCH3;
(ii) n is 1; R₁, R₂, R₃, R₄ are ethyl; and R₅ is hydrogen or CH2CHOHCH3;
(iii) n is 2; each R₁ and R₂ is ethyl; R₃ and R₄ is ethyl; and R₅ is hydrogen or CH2CHOHCH3;
(iv) n is 3; each R₁ and R₂ is ethyl; R₃ and R₄ is ethyl; and R₅ is hydrogen or CH2CHOHCH3.

13. The method of any preceding claim, wherein the surface extender is dextran;
optionally wherein the dextran has a molecular weight of from about 10 to about 2000 kDa, such as about 40 kDa, and/or
optionally wherein the density of dextran is from about 5 to about 30 mg dextran per ml of the strong anion exchange chromatography material.

14. The method of any preceding claim, wherein the adeno-associated virus capsids are capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof;
optionally wherein the adeno-associated virus capsids are capsids of adeno-associated virus serotype 9 (AAV9) or a variant thereof.

15. The method of any one of claims 6-10, wherein the chromatography material is defined by Formula IV: 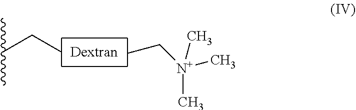
wherein the elution buffer of step (b) of claim 1 and the elution buffer of steps (f) and (g) of claim 6 comprise sodium acetate,
wherein the adeno-associated virus capsids are capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof;
optionally wherein the adeno-associated virus capsids are capsids of adeno-associated virus serotype 9 (AAV9) or a variant thereof.

16. The method of claim 11, wherein the chromatography material is defined by Formula IV: 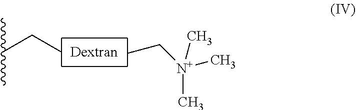
wherein the elution buffer of steps (II) and (III) of claim 11 comprises sodium acetate;
wherein the adeno-associated virus capsids are capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof;
optionally wherein the adeno-associated virus capsids are capsids of adeno-associated virus serotype 9 (AAV9) or a variant thereof.

17. Use of an anion exchange chromatography material comprising a support, a ligand, and a surface extender connecting the ligand to the support, and being defined by Formula IV: 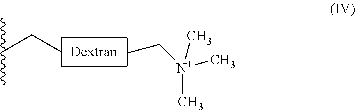
for separating adeno-associated virus capsids fully packaged with genetic material from adeno-associated virus capsids not fully packaged with genetic material,
comprising determining elution conditions suitable for separating adeno-associated virus capsids fully packaged with genetic material from adeno-associated virus capsids not fully packaged with genetic material,
wherein said elution conditions are determined by performing the steps:
a. adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, to the chromatography material;
b. eluting the adeno-associated virus capsids not fully packaged with genetic material and the adeno-associated virus capsids fully packaged with genetic material from the chromatography material by applying an elution buffer comprising a step gradient of increasing conductivity, which starts at from about 0 to about 5 mS/cm, and which increases by from about 0.5 to about 3 mS/cm per step, such as from about 1 to about 2 mS/cm per step, such as from about 1.2 to about 1.5 mS/cm per step, at least up to and including a conductivity at which the adeno-associated virus capsids not fully packaged with genetic material and the adeno-associated virus capsids fully packaged with genetic material have been eluted from the chromatography material;
c. based on an elution profile obtained in step (b), determining a first value of conductivity or conductivity-related parameter, which is suitable for eluting the adeno-associated virus capsids not fully packaged with genetic material, and
d. based on the elution profile obtained in step (b), determining a second value of conductivity or conductivity-related parameter, which is suitable for eluting the adeno-associated virus capsids fully packaged with genetic material.

18. The use of claim 17, wherein the adeno-associated virus capsids fully packaged with genetic material are separated from adeno-associated virus capsids not fully packaged with genetic material by performing steps (a)-(d) of claim 17 and further by performing the steps:
e. adding a liquid sample comprising adeno-associated virus capsids of a purity of at least 90% and of a concentration of at least 10¹² adeno-associated virus capsids/ml, of which at least 5%, such as 10%, of the adeno-associated virus capsids are adeno-associated virus capsids fully packaged with genetic material, the liquid sample originating from a cell culture harvest from which the liquid sample of step (a) originated, to the chromatography material;
f. eluting the adeno-associated virus capsids not fully packaged with genetic material by applying an elution buffer having the first value of conductivity or conductivity-related parameter as determined in step (c); and
g. eluting the adeno-associated virus capsids fully packaged with genetic material by applying an elution buffer having the second value of conductivity or conductivity-related parameter as determined in step (d);
wherein
(i) the duration of step (f) is at least 3 times, such as 4 times, the duration of step (g), and/or
(ii) the method comprises a step (f′) between step (f) and step (g), wherein step (f′) comprises applying an additional step elution and/or a gradient of increasing conductivity between the first value and the second value of conductivity or conductivity-related parameter, and the duration of steps (f) and (f′) is at least 3 times, such as 4 times, the duration of step (g).

19. The use of claim 17 or 18, wherein the elution buffer of step (b) of claim 17, and when referring to claim 18 also the elution buffer of steps (f) and (g) of claim 18, comprises sodium acetate;
wherein the adeno-associated virus capsids are capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof;
optionally wherein the adeno-associated virus capsids are capsids of adeno-associated virus serotype 9 (AAV9) or a variant thereof.
